(19) United States
(12) Patent Application Publication
(10) Pub. No.: US 2014/0286946 A1
(43) Pub. Date: Sep. 25, 2014

(54) METHOD FOR PREPARING ANTIBODIES HAVING IMPROVED PROPERTIES

(71) Applicant: Merck Sharp & Dohme Corp., Rahway, NJ (US)

(72) Inventors: Terrance A. Stadheim, Lyme, NH (US); Daniel Cua, Palo Alto, CA (US); Dongxing Zha, Etna, NH (US)

(21) Appl. No.: 14/354,931

(22) PCT Filed: Oct. 26, 2012
(86) PCT No.: PCT/US2012/062211

§ 371 Date: Apr. 29, 2014

Related U.S. Application Data
(60) Provisional application No. 61/553,335, filed on Oct. 31, 2011.

(51) Int. Cl. C07K 16/28; C07K 16/24

(52) U.S. Cl. CPC C07K 16/2878; C07K 16/241
USPC 424/133.1

(57) ABSTRACT

The present invention is directed to methods and compositions for the production of Fc-containing polypeptides having improved properties.

### FIELD OF THE INVENTION

The present invention is directed to methods and compositions for the production of Fc-containing polypeptides which are useful as human or animal therapeutic agents.

### BACKGROUND OF THE INVENTION

Therapeutic proteins often achieve their therapeutic benefit through engagement, or binding, to an endogenous protein or physiological component to effect a desired response. For example, monoclonal antibodies often achieve their therapeutic benefit through two binding events. First, the variable domain of the antibody binds a specific protein on a target cell, for example CD20 on the surface of cancer cells. This is followed by recruitment of effector cells such as natural killer (NK) cells that bind to the constant region (Fc) of the antibody and destroy cells to which the antibody is bound. This process, known as antibody-dependent cell cytotoxicity (ADCC), depends on a specific N-glycosylation event at Asn 297 in the Fc domain of the heavy chain of IgG1s, Rothman et al., Mol. Immunol. 26: 1113-1123 (1989). Antibodies that lack this N-glycosylation structure still bind antigen but cannot mediate ADCC, apparently as a result of reduced affinity of the Fc domain of the antibody for the Fc Receptor FcγRIIIa on the surface of NK cells.

The presence of N-glycosylation not only plays a role in the effector function of an antibody, the particular composition of the N-linked oligosaccharide is also important for its end function. The lack of fucose or the presence of bisecting N-acetyl glucosamine has been positively correlated with the potency of the ADCC, Rothman (1989), Umana et al., Nat. Biotech. 17: 176-180 (1999), Shields et al., J. Biol. Chem. 277: 26733-26740 (2002), and Shinkawa et al., J. Biol. Chem. 278: 3466-3473 (2003). There is also evidence that sialylation in the Fc region is positively correlated with the anti-inflammatory properties of intravenous immunoglobulin (IVIG). See, e.g., Kaneko et al., Science, 313: 670-673, 2006; Nimmerjahn and Ravetch., J. Exp. Med., 204: 11-15, 2007.

Given the utility of specific N-glycosylation in the function and potency of antibodies, a method for modifying the composition of N-linked oligosaccharides in antibodies to modify their function would be desirable. In particular, it would be desirable to modify the composition of N-linked oligosaccharides in order to confer to Fc-containing peptides, such as antibodies, an increased or enhanced ability of activating immune cells. Such antibodies could be used to treat infectious diseases or neoplastic diseases as well as to serve as an adjuvant for vaccines.

Yeast and other fungal hosts are important production platforms for the generation of recombinant proteins. Yeasts are eukaryotes and, therefore, share common evolutionary processes with higher eukaryotes, including many of the post-translational modifications that occur in the secretory pathway. Recent advances in glycoengineering have resulted in cell lines of the yeast strain Pichia pastoris with genetically modified glycosylation pathways that allow them to carry out a sequence of enzymatic reactions, which mimic the process of glycosylation in humans. See, for example, U.S. Pat. Nos. 7,029,872, 7,326,681 and 7,449,308 that describe methods for producing a recombinant glycoprotein in a lower eukaryote host cell that are substantially identical to their human counterparts. Human-like sialylated bi-antennary complex N-linked glycans like those produced in yeast from the aforesaid methods have demonstrated utility for the production of therapeutic glycoproteins. Thus, a method for further modifying or improving the production of antibodies in yeasts such as Pichia pastoris would be desirable.

### SUMMARY OF THE INVENTION

The invention comprises a method of enhancing an immune response in a subject in need thereof comprising: administering to the subject a therapeutically effective amount of an Fc-containing polypeptide comprising an increased amount of α-2,3-linked sialic acid compared to the amount of α-2,3-linked in a parent polypeptide. In one embodiment, the subject has, or is at risk of developing, an infectious disease or a neoplastic disease.

In one embodiment, the amount of α-2,3-linked sialic acid is increased (compared to the amount of α-2,3-linked in a parent polypeptide) by introducing one or more mutations in the Fc region of the Fc-containing polypeptide.

In one embodiment, the amount of α-2,3-linked sialic acid is increased (compared to the amount of α-2,3-linked in a parent polypeptide) by expressing the Fc-containing polypeptide in a host cell that has α-2,3 sialic acid transferase. In another embodiment, the amount of α-2,3-linked sialic acid is increased (compared to the amount of α-2,3-linked in a parent polypeptide) by expressing the Fc-containing polypeptide in a host cell that has been transformed with a nucleic acid encoding an α-2,3 sialic acid transferase. In one embodiment the host cell is a mammalian cell. In one embodiment, the host cell is a lower eukaryotic host cell. In one embodiment, the host cell is fungal host cell. In one embodiment, the host cell is Pichia sp. In one embodiment, the host cell is Pichia pastoris.

In one embodiment, the amount of α-2,3-linked sialic acid is increased (compared to the amount of α-2,3-linked in a parent polypeptide) by introducing one or more mutations in the Fc region of the Fc-containing polypeptide and by expressing the Fc-containing polypeptide in a host cell that has been transformed with a nucleic acid encoding an α-2,3 sialic acid transferase.

In one embodiment, the invention comprises a method of enhancing an immune response in a subject in need thereof comprising administering to the subject a therapeutically effective amount of an Fc-containing polypeptide comprising sialylated N-glycans, wherein the sialic acid residues in the sialylated N-glycans contain α-2,3 linkages, and wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃GlcNAc₂. In one embodiment, the sialic acid residues in the sialylated N-glycans are attached exclusively via α-2,3 linkages. In one embodiment, the subject has, or is at risk of developing, an infectious disease or a neoplastic disease. In one embodiment, at least 30%, 40%, 50%, 60%, 70%, 80% or 90% of the N-glycans on the Fc-containing polypeptide comprise an N-linked oligosaccharide structure selected from the group consisting of SA₂Gal₍₁₋₄₎GlcNAc₍₂₋₄₎Man₃GlcNAc₂. In one embodiment, at least 30%, 40%, 50%, 60%, 70%, 80% or 90% of the N-glycans on the Fc-containing polypeptide comprise an N-linked oligosaccharide structure selected from the group consisting of SA₂Gal₂GlcNAc₂Man₃GlcNAc₂. In one embodiment, at least 80% of the N-glycans on the Fc-containing polypeptide comprise an N-linked oligosaccharide structure selected from the group consisting of SA₂Gal₂GlcNAc₂Man₃GlcNAc₂. In any of the above embodiments, the SA could be NANA or NGNA, or an analog or derivative of NANA or NGNA. In one embodiment, at least 30%, 40%, 50%, 60%, 70%, 80% or 90% of the N-glycans on the Fc-containing polypeptide comprise an N-linked oligosaccharide structure selected from the group consisting of NANA₂Gal₂GlcNAc₂Man₃GlcNAc₂. In one embodiment, the N-glycans lack fucose. In another embodiment, the N-glycans further comprise a core fucose.

In one embodiment, the invention comprises a method of enhancing an immune response in a subject in need thereof comprising administering to the subject a therapeutically effective amount of an Fc-containing polypeptide comprising sialylated N-glycans, wherein the sialic acid residues in the sialylated N-glycans contain α-2,3 linkages, and wherein at least 30%, 40%, 50%, 60%, 70%, 80% or 90% of the N-glycans on the Fc-containing polypeptide comprise an N-linked oligosaccharide structure selected from the group consisting of SA(1-4)Gal(1-4)GlcNAc(1-4)Man(>=3)GlcNAc₂. In one embodiment, at least 30%, 40%, 50%, 60%, 70%, 80% or 90% of the N-glycans on the Fc-containing polypeptide comprise an oligosaccharide structure selected from the group consisting of SA(1-3)Gal(1-3)GlcNAc(1-3)Man3GlcNAc2. In any of the above embodiments, the SA could be NANA or NGNA, or an analog or derivative of NANA or NGNA. In one embodiment, the sialic acid residues in the sialylated N-glycans are attached exclusively via α-2,3 linkages.

In one embodiment, the invention comprises a method of treating a neoplastic disease (tumor) in a subject comprising administering to the subject a therapeutically effective amount of an Fc-containing polypeptide comprising sialylated N-glycans, wherein the sialic acid residues in the sialylated N-glycans contain α-2,3 linkages, and wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃GlcNAc₂. In one embodiment, the sialic acid residues in the sialylated N-glycans are attached exclusively via α-2,3 linkages. In one embodiment, the subject has, or is at risk of developing, an infectious disease or a neoplastic disease. In one embodiment, at least 30%, 40%, 50%, 60%, 70%, 80% or 90% of the N-glycans on the Fc-containing polypeptide comprise an N-linked oligosaccharide structure selected from the group consisting of SA₂Gal₍₁₋₄₎GlcNAc₍₂₋₄₎Man₃GlcNAc₂. In one embodiment, at least 30%, 40%, 50%, 60%, 70%, 80% or 90% of the N-glycans on the Fc-containing polypeptide comprise an N-linked oligosaccharide structure selected from the group consisting of SA₂Gal₂GlcNAc₂Man₃GlcNAc₂. In one embodiment, at least 80% of the N-glycans on the Fc-containing polypeptide comprise an N-linked oligosaccharide structure selected from the group consisting of SA₂Gal₂GlcNAc₂Man₃GlcNAc₂. In any of the above embodiments, the SA could be NANA or NGNA, or an analog or derivative of NANA or NGNA. In one embodiment, at least 30%, 40%, 50%, 60%, 70%, 80% or 90% of the N-glycans on the Fc-containing polypeptide comprise an N-linked oligosaccharide consisting of NANA₂Gal₂GlcNAc₂Man₃GlcNAc₂. In one embodiment, the N-glycans lack fucose. In another embodiment, the N-glycans further comprise a core fucose.

In any of the above identified embodiments, the Fc polypeptide can be an antibody or antibody fragment comprising sialylated N-glycans. In one embodiment, the Fc polypeptide comprises N-glycans at a position that corresponds to the Asn297 site of a full-length heavy chain antibody, wherein the numbering is according to the EU index as in Kabat. In one embodiment, the Fc polypeptide is an antibody or antibody fragment comprising or consisting essentially of SEQ ID NO:6 or SEQ ID NO:7. In one embodiment the Fc-containing polypeptide comprises or consists of the amino acid sequence of SEQ ID NO: 6 or SEQ ID NO: 7, plus or more mutations which result in an increased amount of sialic acid when compared to the amount of sialic acid in the parent polypeptide. In one embodiment the Fc-containing polypeptide comprises or consists of the amino acid sequence of SEQ ID NO: 6 or SEQ ID NO: 7, plus one, two, three or four mutations which result in an increased amount of sialic acid when compared to the amount of sialic acid in the parent polypeptide. In one embodiment, the parent polypeptide comprises the amino acid sequence of SEQ ID NO:6 or SEQ ID NO:7. In one embodiment, the Fc-containing polypeptide is an antibody or antibody fragment comprising a mutation at position 243 of the Fc region wherein the numbering is according to EU index as in Kabat. In one embodiment, the mutation is F243A. In one embodiment, the Fc-containing polypeptide is an antibody or antibody fragment comprising a mutation at position 264 of the Fc region wherein the numbering is according to EU index as in Kabat. In one embodiment, the mutation is V264A. In one embodiment, the Fc-containing polypeptide is an antibody or antibody fragment comprising mutations at positions 243 and 264 of the Fc region wherein the numbering is according to EU index as in Kabat. In one embodiment, the mutations are F243A and V264A.

In one embodiment the Fc-containing polypeptide has one or more of the following properties when compared to a parent Fc-containing polypeptide: increased effector function, increased ability to recruit immune cells, and increased inflammatory properties.

The invention also comprises a method of enhancing an immune response in a subject in need thereof comprising: administering to the subject a therapeutically effective amount of an Fc-containing polypeptide comprising N-glycans, wherein at at least 30%, 40%, 50%, 60%, 70%, 80% or 90% of the N-glycans on the Fc-containing polypeptide comprise an oligosaccharide structure selected from the group consisting of SA₍₁₋₄₎Gal₍₁₋₄₎GlcNAc₍₂₋₄₎Man₃GlcNAc₂, In one embodiment, the sialic acid residues are exclusively attached through an α-2,3 linkage. In one embodiment, the subject has, or is at risk of developing, an infectious disease or a neoplastic disease. In one embodiment, at least 30%, 40%, 50%, 60%, 70%, 80% or 90% of the N-glycans on the Fc-containing polypeptide comprise an N-linked oligosaccharide structure selected from the group consisting of SA₂Gal₍₁₋₄₎GlcNAc₍₂₋₄₎Man₃GlcNAc₂. In one embodiment, at least 30%, 40%, 50%, 60%, 70%, 80% or 90% of the N-glycans on the Fc-containing polypeptide comprise an N-linked oligosaccharide structure consisting of SA₂Gal₂GlcNAc₂Man₃GlcNAc₂. In one embodiment, at least 80% of the N-glycans on the Fc-containing polypeptide comprise an N-linked oligosaccharide structure selected from the group consisting of SA₂Gal₂GlcNAc₂Man₃GlcNAc₂. In any of the above embodiments, the SA could be NANA or NGNA, or an analog or derivative of NANA or NGNA. In one embodiment, at least 30%, 40%, 50%, 60%, 70%, 80% or 90% of the N-glycans on the Fc-containing polypeptide comprise an N-linked oligosaccharide structure consisting of NANA₂Gal₂GlcNAc₂Man₃GlcNAc₂. In one embodiment, the N-glycans lack fucose. In another embodiment, the N-glycans further comprise a core fucose.

The invention also comprises a method of enhancing an immune response in a subject in need thereof comprising: administering to the subject a therapeutically effective amount of an Fc-containing polypeptide comprising sialylated N-glycans, wherein the sialic acid residues in the Fc-containing polypeptide contain an α-2,3 linkage, and wherein the Fc-containing polypeptide comprises or consists of the amino acid sequence of SEQ ID NO: 6 or SEQ ID NO: 7, plus one or more mutations which result in an increased amount of sialic acid when compared to the amount of sialic acid in the parent polypeptide. In one embodiment, the Fc-containing polypeptide comprises the amino acid sequence of SEQ ID NO:6 or SEQ ID NO:7, plus one, two, three or four mutations which result in an increased amount of sialic acid when compared to the amount of silaic acid in the parent polypeptide. In one embodiment, the parent polypeptide comprises the amino acid sequence of SEQ ID NO:6 or SEQ ID NO:7. In one embodiment, at least 30%, 40%, 50%, 60%, 70%, 80% or 90% of the N-glycans on the Fc-containing polypeptide comprise an oligosaccharide structure selected from the group consisting of SA₍₁₋₄₎Gal₍₁₋₄₎GlcNAc₍₂₋₄₎Man₃GlcNAc₂. In one embodiment, at least 30%, 40%, 50%, 60%, 70%, 80% or 90% of the N-glycans on the Fc-containing polypeptide comprise an N-linked oligosaccharide structure selected from the group consisting of SA₂Gal₂GlcNAc₂Man₃GlcNAc₂. In one embodiment, at least 80% of the N-glycans on the Fc-containing polypeptide comprise an N-linked oligosaccharide structure selected from the group consisting of SA₂Gal₂GlcNAc₂Man₃GlcNAc₂. In one embodiment, at least 30%, 40%, 50%, 60%, 70%, 80% or 90% of the N-glycans on the Fc-containing polypeptide comprise an N-linked oligosaccharide structure selected from the group consisting of NANA₂Gal₂GlcNAc₂Man₃GlcNAc₂. In one embodiment, the sialic acid residues in the sialylated N-glycans are attached exclusively via α-2,3 linkages.

The invention also comprises a pharmaceutical formulation comprising an Fc-containing polypeptide, wherein the Fc-containing polypeptide comprises sialylated N-glycans, wherein the sialic acid residues in the sialylated N-glycans are attached exclusively via α-2,3 linkages.

The invention also comprises a pharmaceutical formulation comprising an Fc-containing polypeptide, wherein the Fc-containing polypeptide comprises sialylated N-glycans, wherein the sialic acid residues in the sialylated N-glycans contain α-2,3 linkages, and wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃GlcNAc₂. In one embodiment, at least wherein at least 30%, 40%, 50%, 60%, 70%, 80% or 90% of the N-glycans on the Fc-containing polypeptide comprise an N-linked oligosaccharide structure selected from the group consisting of SA₂Gal₍₁₋₄₎GlcNAc₍₂₋₄₎Man₃GlcNAc₂. In one embodiment, at least wherein at least 30%, 40%, 50%, 60%, 70%, 80% or 90% of the N-glycans on the Fc-containing polypeptide comprise an N-linked oligosaccharide structure consisting of SA₂Gal₂GlcNAc₂Man₃GlcNAc₂. In one embodiment, at least 80% of the N-glycans on the Fc-containing polypeptide comprise an N-linked oligosaccharide structure selected from the group consisting of SA₂Gal₂GlcNAc₂Man₃GlcNAc₂. In one embodiment, at least wherein at least 30%, 40%, 50%, 60%, 70%, 80% or 90% of the N-glycans on the Fc-containing polypeptide comprise an N-linked oligosaccharide structure consisting of NANA₂Gal₂GlcNAc₂Man₃GlcNAc₂. In one embodiment, the sialic acid residues in the sialylatd N-glycans are attached exclusively via α-2,3 linkages. In one embodiment, the N-glycans lack fucose. In another embodiment, the N-glycans further comprise a core fucose.

In any one of the embodiments directed to pharmaceutical formulations, the Fc-containing polypeptide can be an antibody or an antibody fragment comprising sialylated N-glycans. In one embodiment, the Fc polypeptide comprises N-glycans at a position that corresponds to the Asn297 site of a full-length heavy chain antibody, wherein the numbering is according to the EU index as in Kabat. In one embodiment, the Fc-containing polypeptide is an antibody or antibody fragment comprising the amino acid sequence of SEQ ID NO:6 or SEQ ID NO:7, plus one or more mutations which result in an increased amount of sialic acid when compared to the amount of silaic acid in the parent polypeptide. In one embodiment, the Fc-containing polypeptide is an antibody or antibody fragment comprising or consisting essentially of the amino acid sequence of SEQ ID NO: 6 or SEQ ID NO: 7, plus one, two, three or four mutations which result in an increased amount of sialic acid when compared to the amount of sialic acid in the parent polypeptide. In one embodiment, the parent polypeptide comprises the amino acid sequence of SEQ ID NO:6 or SEQ ID NO:7. In one embodiment, the Fc-containing polypeptide is an antibody or antibody fragment comprising mutations at positions 243 and 264 of the Fc region wherein the numbering is according to EU index as in Kabat. In one embodiment, the mutations are F243A and V264A. In one embodiment the Fc-containing polypeptide has one or more of the following properties when compared to a parent Fc-containing polypeptide: increased effector function, increased ability to recruit immune cells, and increased inflammatory properties.

### BRIEF DESCRIPTION OF THE DRAWINGS

FIG. 1 shows the anti-tumor efficacy (by reduction in tumor volume) of various antibodies in a 4T1-Luc2 model.

FIG. 2 shows the anti-tumor efficacy (by reduction in tumor volume) of various antibodies in a 4T1-Luc2 model.

FIG. 3 shows the tumor growth inhibition (TGI) of various antibodies in a 4T1-Luc2 model.

FIG. 4 shows images of cancer metastasis to lung tissue from tumor-implanted mice treated with various antibodies.

FIG. 5 shows the effect of alpha2,3 sialylated Fc in an AIA model as described in Example 4.

### DETAILED DESCRIPTION OF THE INVENTION

### Definitions

The term “G0” when used herein refers to a complex bi-antennary oligosaccharide without galactose or fucose, GlcNAc₂Man₃GlcNAc₂.

The term “G1” when used herein refers to a complex bi-antennary oligosaccharide without fucose and containing one galactosyl residue, GalGlcNAc₂Man₃GlcNAc₂.

The term “G2” when used herein refers to a complex bi-antennary oligosaccharide without fucose and containing two galactosyl residues, Gal₂GlcNAc₂Man₃GlcNAc₂.

The term “G0F” when used herein refers to a complex bi-antennary oligosaccharide containing a core fucose and without galactose, GlcNAc₂Man₃GlcNAc₂F.

The term “G1F” when used herein refers to a complex bi-antennary oligosaccharide containing a core fucose and one galactosyl residue, GalGlcNAc₂Man₃GlcNAc₂F.

The term “G2F” when used herein refers to a complex bi-antennary oligosaccharide containing a core fucose and two galactosyl residues, Gal₂GlcNAc₂Man₃GlcNAc₂F.

The term “Man5” when used herein refers to the oligosaccharide structure shown as

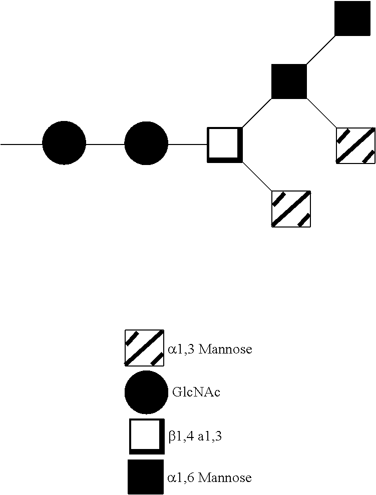

The term “GFI 5.0” when used herein refers to glycoengineered Pichia pastoris strains that produce glycoproteins having predominantly Gal₂GlcNAc₂Man₃GlcNAc₂ N-glycans.

The term “GFI 6.0” when used herein refers to glycoengineered Pichia pastoris strains that produce glycoproteins having predominantly SA₂Gal₂GlcNAc₂Man₃GlcNAc₂ N-glycans.

The term “GS5.0”, when used herein refers to the N-glycosylation structure Gal₂GlcNAc₂Man₃GlcNAc₂.

The term “GS5.5”, when used herein refers to the N-glycosylation structure SAGal₂GlcNAc₂Man₃GlcNAc₂, which when produced in Pichia pastoris strains to which α-2,6 sialyl transferase has been glycoengineered result in α-2,6-linked sialic acid, which when produced in Pichia pastoris strains to which α-2,3 sialyl transferase has been glycoengineered result in α-2,3-linked sialic acid, and which when produced in Pichia pastoris strains to which α-2,6 sialyl transferase and α-2,3 sialyl transferase have been glycoengineered result in a mixture of α-2,6- and α-2,3-linked sialic acid species. The sialic acid produced in Pichia pastoris is of the N-acetyl neuraminic acid (NANA) type unless the strain has been engineered to express CMP-NANA hydroxylase wherein the sialic acid will be a mixture of N-glycolyl neuraminic acid (NGNA) and NANA.

The term “GS6.0”, when used herein refers to the N-glycosylation structure SA₂Gal₂GlcNAc₂Man₃GlcNAc₂, which when produced in Pichia pastoris strains to which α-2,6 sialyl transferase has been glycoengineered result in α-2,6-linked sialic acid and which when produced in Pichia pastoris strains to which α-2,3 sialyl transferase has been glycoengineered result in α-2,3-linked sialic acid, and which when produced in Pichia pastoris strains to which α-2,6 sialyl transferase and α-2,3 sialyl transferase have been glycoengineered result in a mixture of α-2,6- and α-2,3-linked sialic acid species. The sialic acid produced in Pichia pastoris is of the N-acetyl neuraminic acid (NANA) type unless the strain has been engineered to express CMP-NANA hydroxylase wherein the sialic acid will be a mixture of N-glycolyl neuraminic acid (NGNA) and NANA.

The term “wild type” or “wt” when used herein in connection to a Pichia pastoris strain refers to a native Pichia pastoris strain that has not been subjected to genetic modification to control glycosylation.

The term “antibody”, when used herein refers to an immunoglobulin molecule capable of binding to a specific antigen through at least one antigen recognition site located in the variable region of the immunoglobulin molecule. As used herein, the term encompasses not only intact polyclonal or monoclonal antibodies, consisting of four polypeptide chains, i.e. two identical pairs of polypeptide chains, each pair having one “light” chain (LC) (about 25 kDa) and one “heavy” chain (HC) (about 50-70 kDa), but also fragments thereof, such as Fab, Fab′, F(ab′)₂, Fv, single chain (ScFv), mutants thereof, bispecific formats, fusion proteins comprising an antibody portion, and any other modified configuration of an immunoglobulin molecule that comprises an antigen recognition site and at least the portion of the C_{H}2 domain of the heavy chain immunoglobulin constant region which comprises an N-linked glycosylation site of the C_{H}2 domain, or a variant thereof. As used herein the term includes an antibody of any class, such as IgG (for example, IgG1, IgG2, IgG3 or IgG4), IgM, IgA, IgD and IgE, respectively.

The term “consensus sequence of C_{H}2” when used herein refers to the amino acid sequence of the C_{H}2 domain of the heavy chain constant region containing an N-linked glycosylation site which was derived from the most common amino acid sequences found in C_{H}2 domains from a variety of antibodies.

The term “Fc region” is used to define a C-terminal, or so-called effector region, of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The Fc region of an immunoglobulin comprises two constant domains, CH2 and CH3, and can optionally comprise a hinge region. In one embodiment, the Fc region comprises the amino acid sequence of SEQ ID NO:6. In one embodiment, the Fc region comprises the amino acid sequence of SEQ ID NO:7. In another embodiment, the Fc region comprises the amino acid sequence of SEQ ID NO:6, with the addition of a lysine (K) residue at the 3′ end. The Fc region contains a single N-linked glycosylation site in the CH2 domain that corresponds to the Asn297 site of a full-length heavy chain of an antibody, wherein the numbering is according to the EU index as in Kabat.

The term “Fc-containing polypeptide” refers to a polypeptide, such as an antibody or immunoadhesin, which comprises an Fc region or a fragment of an Fc region which retains the N-linked glycosylation site in the CH2 domain and retains the ability to recruit immune cells. This term encompasses polypeptides comprising or consisting of (or consisting essentially of) an Fc region either as a monomer or dimeric species. Polypeptides comprising an Fc region can be generated by papain digestion of antibodies or by recombinant DNA technology.

The term “parent antibody”, “parent immunoglobulin” or “parent Fc-containing polypeptide” when used herein refers to an antibody or Fc-containing polypeptide which lacks the Fc region mutations disclosed herein. A parent Fc-containing polypeptide may comprise a native sequence Fc region or an Fc region with pre-existing amino acid sequence modifications. A native sequence Fc region comprises an amino acid sequence identical to the amino acid sequence of an Fc region found in nature. Native sequence Fc regions include the native sequence human IgG1 Fc region, the native sequence human IgG2 Fc region, the native sequence human IgG3 Fc region and the native sequence human IgG4 Fc region as well as naturally occurring variants thereof. When used as a comparator, a parent antibody or a parent Fc-containing polypeptide can be expressed in any cell. In one embodiment, the parent antibody or a parent Fc-containing polypeptide is expressed in the same cell as the Fc-containing polypeptide of the invention.

As used herein, the term “immunoadhesin” designates antibody-like molecules which combine the “binding domain” of a heterologous “adhesin” protein (e.g. a receptor, ligand or enzyme) with an immunoglobulin constant domain. Structurally, the immunoadhesins comprise a fusion of the adhesin amino acid sequence with the desired binding specificity which is other than the antigen recognition and binding site (antigen combining site) of an antibody (i.e. is “heterologous”) and an immunoglobulin constant domain sequence. The term “ligand binding domain” as used herein refers to any native cell-surface receptor or any region or derivative thereof retaining at least a qualitative ligand binding ability of a corresponding native receptor. In a specific embodiment, the receptor is from a cell-surface polypeptide having an extracellular domain that is homologous to a member of the immunoglobulin supergenefamily. Other receptors, which are not members of the immunoglobulin supergenefamily but are nonetheless specifically covered by this definition, are receptors for cytokines, and in particular receptors with tyrosine kinase activity (receptor tyrosine kinases), members of the hematopoietin and nerve growth factor which predispose the mammal to the disorder in question. In one embodiment, the disorder is cancer. Methods of making immunoadhesins are well known in the art. See, e.g., WO00/42072.

The term “Fc mutein antibody” when used herein refers to an antibody comprising one or more mutations in the Fc region.

The term “Fc mutein” when used herein refers to an Fc-containing polypeptide in which one or more point mutations have been made to the Fc region.

The term “Fc mutation” when used herein refers to a mutation made to the Fc region of an Fc-containing polypeptide. Examples of such a mutation include the F243A or V264A mutations (wherein the numbering is according to EU index as in Kabat). For example, the term “F243A” refers to a mutation from F (wild-type) to A at position 243 of the Fc region of an Fc-containing polypeptide. The term “V264A” refers to a mutation from V (wild-type) to A at position 264 of the Fc region of an Fc-containing polypeptide. The position 243 and 264 represent the amino acid positions in the CH2 domain of the Fc region of an Fc-containing polypeptide. The term “double Fc mutein” when used herein refers to an Fc-containing polypeptide comprising mutations F243A and V264A.

Throughout the present specification and claims, the numbering of the residues in an immunoglobulin heavy chain or an Fc-containing polypeptide is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

The term “effector function” as used herein refers to a biochemical event that results from the interaction of an antibody Fc region with an Fc receptor or ligand. Exemplary “effector functions” include C1q binding; complement dependent cytotoxicity (CDC); Fc receptor binding; antibody-dependent cell-mediated cytotoxicity (ADCC); antibody-dependent cellular phagocytosis (ADCP); phagocytosis; down regulation of cell surface receptors (e. g. B cell receptor; BCR), etc. Such effector functions can be assessed using various assays known in the art.

The term “glycoengineered Pichia pastoris” when used herein refers to a strain of Pichia pastoris that has been genetically altered to express human-like N-glycans. For example, the GFI 5.0, GFI 5.5 and GFI 6.0 strains described above.

The terms “N-glycan”, “glycoprotein” and “glycoform” when used herein refer to an N-linked oligosaccharide, e.g., one that is attached by an asparagine-N-acetylglucosamine linkage to an asparagine residue of a polypeptide. Predominant sugars found on glycoproteins are galactose, mannose, fucose, N-acetylgalactosamine (GalNAc), N-acetylglucosamine (GlcNAc) and sialic acid (Sia or SA, including NANA, NGNA and derivatives and analogs thereof, including acetylated NANA or acetylated NGNA). In glycoengineered Pichia pastoris, sialic acid is exclusively N-acetyl-neuraminic acid (NANA) (Hamilton et al., Science 313 (5792): 1441-1443 (2006)) unless the strains are further engineered to express CMP-NANA hydroxylase to convert NANA into NGNA. N-glycans have a common pentasaccharide core of Man₃GlcNAc₂, wherein “Man” refers to mannose, “Glc” refers to glucose, “NAc” refers to N-acetyl, and GlcNAc refers to N-acetylglucosamine. N-glycans differ with respect to the number of branches (antennae) comprising peripheral sugars (e.g., GlcNAc, galactose, fucose and sialic acid) that are added to the Man₃GlcNAc₂ (“Man3”) core structure which is also referred to as the “trimannose core”, the “pentasaccharide core” or the “paucimannose core”. N-glycans are classified according to their branched constituents (e.g., high mannose, complex or hybrid).

As used herein, the term “sialic acid” or “SA” or “Sia” refers to any member of the sialic acid family, including without limitation: N-acetylneuraminic acid (Neu5Ac or NANA), N-glycolylneuraminic acid (NGNA) and any analog or derivative thereof (including those arising from acetylation at any position on the sialic acid molecule). Sialic acid is a generic name for a group of about 30 naturally occurring acidic carbohydrates that are essential components of a large number of glycoconjugates. Schauer, Biochem. Society Transactions, 11, 270-271 (1983). Sialic acids typically reside at the nonreducing, or terminal, end of oligosaccharides. In humans, sialic acids are usually the terminal residue of the oligosaccharides. N-acetylneuraminic acid (NANA) is the most common sialic acid form and N-glycolylneuraminic acid (NGNA) is the second most common form. Schauer, Glycobiology, 1, 449-452 (1991). NGNA is widespread throughout the animal kingdom and, according to species and tissue, often constitutes a significant proportion of the glycoconjugate-bound sialic acid. Certain species such as chicken and man are exceptional, since they lack NGNA in normal tissues. Corfield, et al., Cell Biology Monographs, 10, 5-50 (1982). In human serum samples, the percentage of sialic acid in the form of NGNA is reported to be 0.01% of the total sialic acid. Schauer, “Sialic Acids as Antigenic Determinants of Complex Carbohydrates”, found in The Molecular Immunology of Complex Carbohydrates, (Plenum Press, New York, 1988).

The term “human-like N-glycan”, as used herein, refers to N-linked oligosaccharides which closely resemble the oligosaccharides produced by non-engineered, wild-type human cells. For example, wild-type Pichia pastoris and other lower eukaryotic cells typically produce hypermannosylated proteins at N-glycosylation sites. The host cells described herein produce glycoproteins (for example, antibodies) comprising human-like N-glycans that are not hypermannosylated. In some embodiments, the host cells of the present invention are capable of producing human-like N-glycans with hybrid and/or complex N-glycans. The specific type of “human-like” glycans present on a specific glycoprotein produced from a host cell of the invention will depend upon the specific glycoengineering steps that are performed in the host cell.

The term “high mannose” type N-glycan when used herein refers to an N-glycan having five or more mannose residues.

The term “complex” type N-glycan when used herein refers to an N-glycan having at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). As an example, when a N-glycan comprises a bisecting GlcNAc on the trimannose core, the structure can be represented as Man₃GlcNAc₂(GlcNAc) or Man₃GlcNAc₃. When an N-glycan comprises a core fucose attached to the trimannose core, the structure may be represented as Man₃GlcNAc₂(Fuc). Complex N-glycans may also have multiple antennae on the “trimannose core,” often referred to as “multiple antennary glycans.”

The term “hybrid” N-glycan when used herein refers to an N-glycan having at least one GlcNAc on the nonreducing terminus of the 1,3 mannose arm of the trimannose core and zero or more than one additional mannose on the nonreducing terminus of the 1,6 mannose arm of the trimannose core.

When referring to “mole percent” of a glycan present in a preparation of a glycoprotein, the term means the molar percent of a particular glycan present in the pool of N-linked oligosaccharides released when the protein preparation is treated with PNGase and then quantified by a method that is not affected by glycoform composition, (for instance, labeling a PNGase released glycan pool with a fluorescent label such as 2-aminobenzamide and then separating by high performance liquid chromatography or capillary electrophoresis and then quantifying glycans by fluorescence intensity). For example, 50 mole percent NANA₂ Gal₂GlcNAc₂Man₃GlcNAc₂ means that 50 percent of the released glycans are NANA₂ Gal₂GlcNAc₂Man₃GlcNAc₂ and the remaining 50 percent are comprised of other N-linked oligosaccharides.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are listed below:

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

Glycosylation of immunoglobulin G (IgG) in the Fc region, Asn297 (according to the EU numbering system), has been shown to be a requirement for optimal recognition and activation of effector pathways including antibody dependent cellular cytotoxicity (ADCC) and complement dependent cytotoxicity (CDC), Wright & Morrison, Trends in Biotechnology, 15: 26-31 (1997), Tao & Morrison, J. Immunol., 143(8):2595-2601 (1989). As such, glycosylation engineering in the constant region of IgG has become an area of active research for the development of therapeutic monoclonal antibodies (mAbs). It has been established that the presence of N-linked glycosylation at Asn297 is critical for mAb activity in immune effector function assays including ADCC, Rothman (1989), Lifely et al., Glycobiology, 5:813-822 (1995), Umana (1999), Shields (2002), and Shinkawa (2003), and complement dependent cytotoxicity (CDC), Hodoniczky et al., Biotechnol. Prog., 21(6): 1644-1652 (2005), and Jefferis et al., Chem. Immunol., 65: 111-128 (1997). This effect on function has been attributed to the specific conformation adopted by the glycosylated Fc domain, which appears to be lacking when glycosylation is absent. More specifically, IgG which lacks glycosylation in the Fc C_{H}2 domain does not bind to FcγR, including FcγRI, FcγRII, and FcγRIII, Rothman (1989).

Not only does the presence of glycosylation appear to play a role in the effector function of an antibody, the particular composition of the N-linked oligosaccharide is also important. For example, the presence of fucose shows a marked effect on in vitro FcγRIIIa binding and in vitro ADCC, Rothman (1989), and Li et al., Nat. Biotechnol. 24(2): 2100-215 (2006). Recombinant antibodies produced by mammalian cell culture, such as CHO or NSO, contain N-linked oligosaccharides that are predominately fucosylated, Hossler et al., Biotechnology and Bioengineering, 95(5):946-960 (2006), Umana (1999), and Jefferis et al., Biotechnol. Prog. 21:11-16 (2005). Additionally, there is evidence that sialylation in the Fc region may impart anti-inflammatory properties to antibodies. Intravenous immunoglobulin (WIG) purified over a lectin column to enrich for the sialylated form showed a distinct anti-inflammatory effect limited to the sialylated Fc fragment and was linked to an increase in expression of the inhibitory receptor FcγRIIb, Nimmerjahn and Ravetch., J. Exp. Med. 204:11-15 (2007).

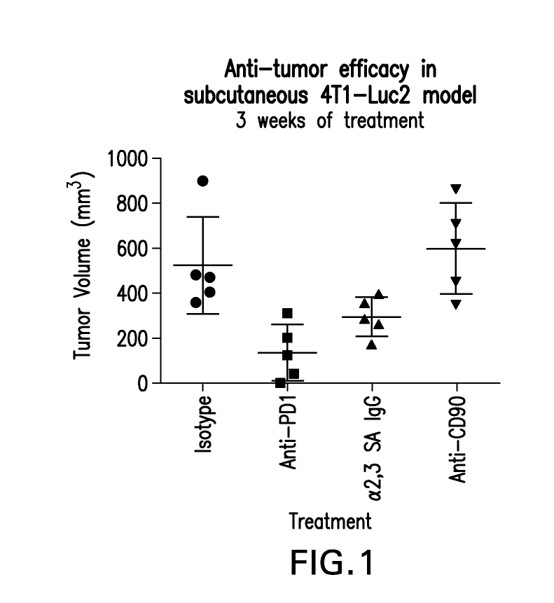

Glycosylation in the Fc region of an antibody derived from mammalian cell lines typically consists of a heterogeneous mix of glycoforms, with the predominant forms typically being comprised of the complex fucosylated glycoforms: G0F, G1F, and, to a lesser extent, G2F. Possible conditions resulting in incomplete galactose transfer to the G0F structure include, but are not limited to, non-optimized galactose transfer machinery, such as β-1,4 galactosyl transferase, and poor UDP-galactose transport into the Golgi apparatus, suboptimal cell culture and protein expression conditions, and steric hindrance by amino acid residues neighboring the oligosaccharide. While each of these conditions may modulate the ultimate degree of terminal galactose, it is thought that subsequent sialic acid transfer to the Fc oligosaccharide is inhibited by the closed pocket configuration of the C_{H}2 domain. See, for example, FIG. 1, Jefferis, R., Nature Biotech., 24 (10): 1230-1231, 2006. Without the correct terminal monosaccharide, specifically galactose, or with insufficient terminal galactosylated forms, there is little possibility of producing a sialylated form, capable of acting as a therapeutic protein, even when produced in the presence of sialyl transferase. Protein engineering and structural analysis of human IgG-Fc glycoforms has shown that glycosylation profiles are affected by Fc conformation, such as the finding that increased levels of galactose and sialic acid on oligosaccharides derived from CHO-produced IgG3 could be achieved when specific single amino acid mutations in the Fc pocket were mutated, to an alanine including F241, F243, V264, D265 and R301. Lund et al., J. Immunol. 157(11); 4963-4969 (1996). It was further shown that certain mutations had some effect on cell-mediated superoxide generation and complement mediated red cell lysis, which are used as surrogate markers for FcγRI and C1q binding, respectively.

Yeast have been genetically engineered to produce host strains capable of secreting glycoproteins with highly uniform glycosylation. Choi et al., PNAS, USA 100(9): 5022-5027 (2003) describes the use of libraries of a 1,2 mannosidase catalytic domains and N-acetylglucosaminyltransferase I catalytic domains in combination with a library of fungal type II membrane protein leader sequences to localize the catalytic domains to the secretory pathway. In this way, strains were isolated that produced in vivo glycoproteins with uniform Man₅GlcNAc₂ or GlcNAcMan5GlcNAc₂ N-glycan structures. Hamilton et al., Science 313 (5792): 1441-1443 (2006) described the production of a glycoprotein, erythropoietin, produced in Pichia pastoris, as having a glycan composition that consisted predominantly of a bisialylated glycan structure, GS6.0, NANA₂Gal₂GlcNAc₂Man₃GlcNAc₂ (90.5%) and monosialylated, GS5.5, NANAGal₂GlcNAc₂ Man₃GlcNAc₂ (7.9%). However, an antibody produced in a similar strain will have a markedly lower content of sialylated N-glycans due to the relatively low level of terminal galactose substrate in the antibody. It has also recently been shown that sialylation of a Fc oligosaccharide imparts anti-inflammatory properties on therapeutic intravenous gamma globulin and its Fc fragments, Kaneko et al., Science 313(5787): 670-673 (2006), and that the anti-inflammatory activity is dependent on the a 2,6-linked but not the α-2,3 linked, form of sialic acid, Anthony et al., Science, 320: 373-376 (2008).

As used herein, the term “neoplastic disease” includes any disease resulting from an abnormal, uncontrolled growth of cells. Neoplasms may be benign, pre-malignant (carcinoma in situ) or malignant (cancer) with or without metastasis or metastatic potential.

As used herein, the term “infectious disease” includes any condition caused by a microorganism or other agent, such as a bacterium, fungus, or virus that enters the body of an organism.

### Host Organisms and Cell Lines

The Fc-containing polypeptides of this invention can be made in any host organism, cell line or in silico. In one embodiment, an Fc-containing polypeptide of the invention is made in a host cell which is capable of producing sialylated N-glycans.

In one embodiment, an Fc-containing polypeptide of the invention is made in a mammalian cell where the cell either endogenously or through genetic or process manipulation produces glycoproteins containing only terminal α-2,3 sialic acid. The propagation of mammalian cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture); baby hamster kidney cells (BHK, ATCC CCL 10); Chinese hamster ovary cells/-DHFR (CHO); mouse sertoli cells (TM4,); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; hybridoma cell lines; NSO; SP2/0; and a human hepatoma line (Hep G2).

In one embodiment, an Fc-containing polypeptide of the invention can be made in a plant cell which is engineered to produce α-2,3 sialylated N-glycans. See, e.g., Cox et al., Nature Biotechnology (2006) 24, 1591-1597 (2006) and Castilho et al., J. Biol. Chem. 285(21): 15923-15930 (2010).

In one embodiment, an Fc-containing polypeptide of the invention can be made in an insect cell which is engineered to produce α-2,3 sialylated N-glycans. See, e.g., Harrison and Jarvis, Adv. Virus Res. 68:159-91 (2006).

In one embodiment, an Fc-containing polypeptide of the invention can be made in a bacterial cell which is engineered to produce α-2,3 sialylated N-glycans. See, e.g., Lizak et al., Bioconjugate Chem. 22:488-496 (2011).

In one embodiment, an Fc-containing polypeptide of the invention can be made in a lower eukaryotic host cell or organism. Recent developments allow for the production of fully humanized therapeutics in lower eukaryotic host organisms, yeast and filamentous fungi, such as Pichia pastoris, Gerngross et al., U.S. Pat. No. 7,029,872 and U.S. Pat. No. 7,449,308, the disclosures of which are hereby incorporated by reference. See also Jacobs et al., Nature Protocols 4(1):58-70 (2009). Applicants herein have further developed modified Pichia pastoris host organisms and cell lines capable of expressing antibodies comprising two mutations to the amino acids at positions 243 and 264 in the Fc region of the heavy chain. The antibodies having these mutations had increased levels and a more homogeneous composition of the α-2,3 linked sialylated N-glycans when compared to a parent antibody.

In one embodiment, an Fc-containing polypeptide of the invention is made in a host cell, more preferably a yeast or filamentous fungal host cell, that has been engineered to produce glycoproteins having a predominant N-glycan comprising a terminal α-2,3-sialic acid. In one embodiment of the invention, the predominant N-glycan is the α-2,3 linked form of SA₂Gal₂GlcNAc₂Man₃GlcNAc₂, produced in strains glycoengineered with α-2,3 sialyl transferase which do not produce any α-2,6 linked sialic acid.

The cell lines to be used to make the Fc-containing polypeptides of the invention can be any cell line, in particular cell lines with the capability of producing one or more α-2,3-sialylated glycoproteins. Those of ordinary skill in the art would recognize and appreciate that the materials and methods described herein are not limited to the specific strain of Pichia pastoris provided as an example herein, but could include any Pichia pastoris strain or other yeast or filamentous fungal strains in which N-glycans with one or more terminal galactose, such as Gal₂GlcNAc₂Man₃, are produced. The terminal galactose acts as a substrate for the production of α-2,3-linked sialic acid, resulting in the N-glycan structure SA₂Gal₂GlcNAc₂Man₃GlcNAc₂. Examples of suitable strains are described in U.S. Pat. No. 7,029,872, U.S. Publication No. 2006-0286637 and Hamilton et al., Science 313 (5792): 1441-1443 (2006), the descriptions of which are incorporated herein as if set forth at length.

In general, lower eukaryotes such as yeast are used for expression of the proteins, particularly glycoproteins because they can be economically cultured, give high yields, and when appropriately modified are capable of suitable glycosylation. Yeast particularly offers established genetics allowing for rapid transformations, tested protein localization strategies and facile gene knock-out techniques. Suitable vectors have expression control sequences, such as promoters, including 3-phosphoglycerate kinase or other glycolytic enzymes, and an origin of replication, termination sequences and the like as desired.

While the invention has been demonstrated herein using the methylotrophic yeast Pichia pastoris, other useful lower eukaryote host cells include Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporiumi lucknowense, Fusarium sp., Fusarium gramineum, Fusarium venenatum, Yarrowia lipotylica and Neurospora crassa. Various yeasts, such as K. lactis, Pichia pastoris, Pichia methanolica, Yarrowia lipolytica and Hansenula polymorpha are particularly suitable for cell culture because they are able to grow to high cell densities and secrete large quantities of recombinant protein. Likewise, filamentous fungi, such as Aspergillus niger, Fusarium sp, Neurospora crassa and others can be used to produce glycoproteins of the invention at an industrial scale.

Lower eukaryotes, particularly yeast and filamentous fungi, can be genetically modified so that they express glycoproteins in which the glycosylation pattern is human-like or humanized. As indicated above, the term “human-like N-glycan”, as used herein refers, to the N-linked oligosaccharides which closely resemble the oligosaccharides produced by non-engineered, wild-type human cells. In preferred embodiments of the present invention, the host cells of the present invention are capable of producing human-like glycoproteins with hybrid and/or complex N-glycans; i.e., “human-like N-glycosylation.” The specific “human-like” glycans predominantly present on glycoproteins produced from the host cells of the invention will depend upon the specific engineering steps that are performed. In this manner, glycoprotein compositions can be produced in which a specific desired glycoform is predominant in the composition. Such can be achieved by eliminating selected endogenous glycosylation enzymes and/or genetically engineering the host cells and/or supplying exogenous enzymes to mimic all or part of the mammalian glycosylation pathway as described in U.S. Pat. No. 7,449,308. If desired, additional genetic engineering of the glycosylation can be performed, such that the glycoprotein can be produced with or without core fucosylation. Use of lower eukaryotic host cells is further advantageous in that these cells are able to produce highly homogenous compositions of glycoprotein, such that the predominant glycoform of the glycoprotein may be present as greater than thirty mole percent of the glycoprotein in the composition. In particular aspects, the predominant glycoform may be present in greater than forty mole percent, fifty mole percent, sixty mole percent, seventy mole percent and, most preferably, greater than eighty mole percent of the glycoprotein present in the composition.

Lower eukaryotes, particularly yeast, can be genetically modified so that they express glycoproteins in which the glycosylation pattern is human-like or humanized. Such can be achieved by eliminating selected endogenous glycosylation enzymes and/or supplying exogenous enzymes as described by Gerngross et al., U.S. Pat. No. 7,449,308. For example, a host cell can be selected or engineered to be depleted in α1,6-mannosyl transferase activities, which would otherwise add mannose residues onto the N-glycan on a glycoprotein.

In one embodiment, the host cell further includes an α1,2-mannosidase catalytic domain fused to a cellular targeting signal peptide not normally associated with the catalytic domain and selected to target the α1,2-mannosidase activity to the ER or Golgi apparatus of the host cell. Passage of a recombinant glycoprotein through the ER or Golgi apparatus of the host cell produces a recombinant glycoprotein comprising a Man₅GlcNAc₂ glycoform, for example, a recombinant glycoprotein composition comprising predominantly a Man₅GlcNAc₂ glycoform. For example, U.S. Pat. Nos. 7,029,872 and 7,449,308 and U.S. Published Patent Application No. 2005/0170452 disclose lower eukaryote host cells capable of producing a glycoprotein comprising a Man₅GlcNAc₂ glycoform.

In a further embodiment, the immediately preceding host cell further includes a GlcNAc transferase I (GnT I) catalytic domain fused to a cellular targeting signal peptide not normally associated with the catalytic domain and selected to target GlcNAc transferase I activity to the ER or Golgi apparatus of the host cell. Passage of the recombinant glycoprotein through the ER or Golgi apparatus of the host cell produces a recombinant glycoprotein comprising a GlcNAcMan₅GlcNAc₂ glycoform, for example a recombinant glycoprotein composition comprising predominantly a GlcNAcMan₅GlcNAc₂ glycoform. U.S. Pat. Nos. 7,029,872 and 7,449,308 and U.S. Published Patent Application No. 2005/0170452 disclose lower eukaryote host cells capable of producing a glycoprotein comprising a GlcNAcMan₅GlcNAc₂ glycoform. The glycoprotein produced in the above cells can be treated in vitro with a hexosaminidase to produce a recombinant glycoprotein comprising a Man₅GlcNAc₂ glycoform.

In a further embodiment, the immediately preceding host cell further includes a mannosidase II catalytic domain fused to a cellular targeting signal peptide not normally associated with the catalytic domain and selected to target mannosidase II activity to the ER or Golgi apparatus of the host cell. Passage of the recombinant glycoprotein through the ER or Golgi apparatus of the host cell produces a recombinant glycoprotein comprising a GlcNAcMan₃GlcNAc₂ glycoform, for example a recombinant glycoprotein composition comprising predominantly a GlcNAcMan₃GlcNAc₂ glycoform. U.S. Pat. No. 7,029,872 and U.S. Published Patent Application No. 2004/0230042 discloses lower eukaryote host cells that express mannosidase II enzymes and are capable of producing glycoproteins having predominantly a GlcNAcMan₃GlcNAc₂ glycoform. The glycoprotein produced in the above cells can be treated in vitro with a hexosaminidase to produce a recombinant glycoprotein comprising a Man₃GlcNAc₂ glycoform.

In a further embodiment, the immediately preceding host cell further includes GlcNAc transferase II (GnT II) catalytic domain fused to a cellular targeting signal peptide not normally associated with the catalytic domain and selected to target GlcNAc transferase II activity to the ER or Golgi apparatus of the host cell. Passage of the recombinant glycoprotein through the ER or Golgi apparatus of the host cell produces a recombinant glycoprotein comprising a GlcNAc₂Man₃GlcNAc₂ glycoform, for example a recombinant glycoprotein composition comprising predominantly a GlcNAc₂Man₃GlcNAc₂ glycoform. U.S. Pat. Nos. 7,029,872 and 7,449,308 and U.S. Published Patent Application No. 2005/0170452 disclose lower eukaryote host cells capable of producing a glycoprotein comprising a GlcNAc₂Man₃GlcNAc₂ glycoform. The glycoprotein produced in the above cells can be treated in vitro with a hexosaminidase to produce a recombinant glycoprotein comprising a Man₃GlcNAc₂ glycoform.

In a further embodiment, the immediately preceding host cell further includes a galactosyltransferase catalytic domain fused to a cellular targeting signal peptide not normally associated with the catalytic domain and selected to target galactosyltransferase activity to the ER or Golgi apparatus of the host cell. Passage of the recombinant glycoprotein through the ER or Golgi apparatus of the host cell produces a recombinant glycoprotein comprising a GalGlcNAc₂ Man₃GlcNAc₂ or Gal₂GlcNAc₂Man₃GlcNAc₂ glycoform, or mixture thereof for example a recombinant glycoprotein composition comprising predominantly a GalGlcNAc₂Man₃GlcNAc₂ glycoform or Gal₂GlcNAc₂Man₃GlcNAc₂ glycoform or mixture thereof. U.S. Pat. No. 7,029,872 and U.S. Published Patent Application No. 2006/0040353 discloses lower eukaryote host cells capable of producing a glycoprotein comprising a Gal₂GlcNAc₂ Man₃GlcNAc₂ glycoform. The glycoprotein produced in the above cells can be treated in vitro with a galactosidase to produce a recombinant glycoprotein comprising a GlcNAc₂Man₃ GlcNAc₂ glycoform, for example a recombinant glycoprotein composition comprising predominantly a GlcNAc₂Man₃GlcNAc₂ glycoform.

In a further embodiment, the immediately preceding host cell further includes a sialyltransferase catalytic domain fused to a cellular targeting signal peptide not normally associated with the catalytic domain and selected to target sialyltransferase activity to the ER or Golgi apparatus of the host cell. In a preferred embodiment, the sialyltransferase is an α-2,3-sialyltransferase. Passage of the recombinant glycoprotein through the ER or Golgi apparatus of the host cell produces a recombinant glycoprotein comprising predominantly a NANA₂Gal₂GlcNAc₂Man₃GlcNAc₂ glycoform or NANAGal₂GlcNAc₂Man₃GlcNAc₂ glycoform or mixture thereof. For lower eukaryote host cells such as yeast and filamentous fungi, it is useful that the host cell further include a means for providing CMP-sialic acid for transfer to the N-glycan. U.S. Published Patent Application No. 2005/0260729 discloses a method for genetically engineering lower eukaryotes to have a CMP-sialic acid synthesis pathway and U.S. Published Patent Application No. 2006/0286637 discloses a method for genetically engineering lower eukaryotes to produce sialylated glycoproteins. To enhance the amount of sialylation, it can be advantageous to construct the host cell to include two or more copies of the CMP-sialic acid synthesis pathway or two or more copies of the sialylatransferase. The glycoprotein produced in the above cells can be treated in vitro with a neuraminidase to produce a recombinant glycoprotein comprising predominantly a Gal₂GlcNAc₂Man₃GlcNAc₂ glycoform or GalGlcNAc₂Man₃GlcNAc₂ glycoform or mixture thereof.

Any one of the preceding host cells can further include one or more GlcNAc transferase selected from the group consisting of GnT III, GnT IV, GnT V, GnT VI, and GnT IX to produce glycoproteins having bisected (GnT III) and/or multiantennary (GnT IV, V, VI, and IX) N-glycan structures such as disclosed in U.S. Published Patent Application Nos. 2005/0208617 and 2007/0037248. Further, the proceeding host cells can produce recombinant glycoproteins (for example, antibodies) comprising SA(1-4)Gal(1-4)GlcNAc(2-4) Man₃GlcNAc₂, including antibodies comprising NANA (1-4)Gal(1-4)GlcNAc(2-4) Man₃GlcNAc₂, NGNA(1-4)Gal(1-4)GlcNAc(2-4)Man₃GlcNAc₂ or a combination of NANA (1-4)Gal(1-4)GlcNAc(2-4) Man₃GlcNAc₂ and NGNA(1-4)Gal(1-4)GlcNAc(2-4) Man₃GlcNAc₂. In one embodiment, the recombinant glycoprotein will comprise N-glycans comprising a structure selected from the group consisting of SA(1-4)Gal(1-4)GlcNAc(2-4) Man₃GlcNAc₂ and devoid of any α-2,6 linked SA.

In further embodiments, the host cell that produces glycoproteins that have predominantly GlcNAcMan₅GlcNAc₂ N-glycans further includes a galactosyltransferase catalytic domain fused to a cellular targeting signal peptide not normally associated with the catalytic domain and selected to target the galactosyltransferase activity to the ER or Golgi apparatus of the host cell. Passage of the recombinant glycoprotein through the ER or Golgi apparatus of the host cell produces a recombinant glycoprotein comprising predominantly the GalGlcNAcMan₅GlcNAc₂ glycoform.

In a further embodiment, the immediately preceding host cell that produced glycoproteins that have predominantly the GalGlcNAcMan₅GlcNAc₂ N-glycans further includes a sialyltransferase catalytic domain fused to a cellular targeting signal peptide not normally associated with the catalytic domain and selected to target sialyltransferase activity to the ER or Golgi apparatus of the host cell. Passage of the recombinant glycoprotein through the ER or Golgi apparatus of the host cell produces a recombinant glycoprotein comprising a SAGalGlcNAcMan₅GlcNAc₂ glycoform (for example NANAGalGlcNAcMan₅GlcNAc₂ or NGNAGalGlcNAcMan₅GlcNAc₂ or a mixture thereof).

Any of the preceding host cells can further include one or more sugar transporters such as UDP-GlcNAc transporters (for example, Kluyveromyces lactis and Mus musculus UDP-GlcNAc transporters), UDP-galactose transporters (for example, Drosophila melanogaster UDP-galactose transporter), and CMP-sialic acid transporter (for example, human sialic acid transporter). Because lower eukaryote host cells such as yeast and filamentous fungi lack the above transporters, it is preferable that lower eukaryote host cells such as yeast and filamentous fungi be genetically engineered to include the above transporters.

Further, any of the preceding host cells can be further manipulated to increase N-glycan occupancy. See e.g., Gaulitzek et al., Biotechnol. Bioengin. 103:1164-1175 (2009); Jones et al., Biochim. Biospyhs. Acta 1726:121-137 (2005); WO2006/107990. In one embodiment, any of the preceding host cells can be further engineered to comprise at least one nucleic acid molecule encoding a heterologous single-subunit oligosaccharyltransferase (for example, Leishmania sp. STT3A protein, STT3B protein, STT3C protein, STT3D protein or combinations thereof) and a nucleic acid molecule encoding the heterologous glycoprotein, and wherein the host cell expresses the endogenous host cell genes encoding the proteins comprising the endogenous OTase complex. In one embodiment, any of the preceding host cells can be further engineered to comprise at least one nucleic acid molecule encoding a Leishmania sp. STT3D protein and a nucleic acid molecule encoding the heterologous glycoprotein, and wherein the host cell expresses the endogenous host cell genes encoding the proteins comprising the endogenous OTase complex.

Host cells further include lower eukaryote cells (e.g., yeast such as Pichia pastoris) that are genetically engineered to produce glycoproteins that do not have α-mannosidase-resistant N-glycans. This can be achieved by deleting or disrupting one or more of the β-mannosyltransferase genes (e.g., BMT1, BMT2, BMT3, and BMT4) (See, U.S. Published Patent Application No. 2006/0211085) and glycoproteins having phosphomannose residues by deleting or disrupting one or both of the phosphomannosyl transferase genes PNO1 and MNN4B (See for example, U.S. Pat. Nos. 7,198,921 and 7,259,007), which in further aspects can also include deleting or disrupting the MNN4A gene. Disruption includes disrupting the open reading frame encoding the particular enzymes or disrupting expression of the open reading frame or abrogating translation of RNAs encoding one or more of the β-mannosyltransferases and/or phosphomannosyltransferases using interfering RNA, antisense RNA, or the like. Further, cells can produce glycoproteins with α-mannosidase-resistant N-glycans through the addition of chemical inhibitors or through modification of the cell culture condition. These host cells can be further modified as described above to produce particular N-glycan structures.

Host cells further include lower eukaryote cells (e.g., yeast such as Pichia pastoris) that are genetically modified to control O-glycosylation of the glycoprotein by deleting or disrupting one or more of the protein O-mannosyltransferase (Dol-P-Man:Protein (Ser/Thr) Mannosyl Transferase genes) (PMTs) (See U.S. Pat. No. 5,714,377) or grown in the presence of Pmtp inhibitors and/or an α-mannosidase as disclosed in Published International Application No. WO 2007/061631, or both. Disruption includes disrupting the open reading frame encoding the Pmtp or disrupting expression of the open reading frame or abrogating translation of RNAs encoding one or more of the Pmtps using interfering RNA, antisense RNA, or the like. The host cells can further include any one of the aforementioned host cells modified to produce particular N-glycan structures.

Pmtp inhibitors include but are not limited to a benzylidene thiazolidinediones. Examples of benzylidene thiazolidinediones that can be used are 5-[[3,4-bis(phenylmethoxy) phenyl]methylene]-4-oxo-2-thioxo-3-thiazolidineacetic Acid; 5-[[3-(1-Phenylethoxy)-4-(2-phenylethoxy)]phenyl]methylene]-4-oxo-2-thioxo-3-thiazolidineacetic Acid; and 5-[[3-(1-Phenyl-2-hydroxy)ethoxy)-4-(2-phenylethoxy)]phenyl]methylene]-4-oxo-2-thioxo-3-thiazolidineacetic acid.

In particular embodiments, the function or expression of at least one endogenous PMT gene is reduced, disrupted, or deleted. For example, in particular embodiments the function or expression of at least one endogenous PMT gene selected from the group consisting of the PMT1, PMT2, PMT3, and PMT4 genes is reduced, disrupted, or deleted; or the host cells are cultivated in the presence of one or more PMT inhibitors. In further embodiments, the host cells include one or more PMT gene deletions or disruptions and the host cells are cultivated in the presence of one or more Pmtp inhibitors. In particular aspects of these embodiments, the host cells also express a secreted α-1,2-mannosidase.

PMT deletions or disruptions and/or Pmtp inhibitors control O-glycosylation by reducing O-glycosylation occupancy, that is, by reducing the total number of O-glycosylation sites on the glycoprotein that are glycosylated. The further addition of an α-1,2-mannsodase that is secreted by the cell controls O-glycosylation by reducing the mannose chain length of the O-glycans that are on the glycoprotein. Thus, combining PMT deletions or disruptions and/or Pmtp inhibitors with expression of a secreted α-1,2-mannosidase controls O-glycosylation by reducing occupancy and chain length. In particular circumstances, the particular combination of PMT deletions or disruptions, Pmtp inhibitors, and α-1,2-mannosidase is determined empirically as particular heterologous glycoproteins (Fabs and antibodies, for example) may be expressed and transported through the Golgi apparatus with different degrees of efficiency and thus may require a particular combination of PMT deletions or disruptions, Pmtp inhibitors, and α-1,2-mannosidase. In another aspect, genes encoding one or more endogenous mannosyltransferase enzymes are deleted. This deletion(s) can be in combination with providing the secreted α-1,2-mannosidase and/or PMT inhibitors or can be in lieu of providing the secreted α-1,2-mannosidase and/or PMT inhibitors.

Thus, the control of O-glycosylation can be useful for producing particular glycoproteins in the host cells disclosed herein in better total yield or in yield of properly assembled glycoprotein. The reduction or elimination of O-glycosylation appears to have a beneficial effect on the assembly and transport of whole antibodies and Fab fragments as they traverse the secretory pathway and are transported to the cell surface. Thus, in cells in which O-glycosylation is controlled, the yield of properly assembled antibodies or Fab fragments is increased over the yield obtained in host cells in which O-glycosylation is not controlled.

To reduce or eliminate the likelihood of N-glycans and O-glycans with β-linked mannose residues, which are resistant to α-mannosidases, the recombinant glycoengineered Pichia pastoris host cells are genetically engineered to eliminate glycoproteins having α-mannosidase-resistant N-glycans by deleting or disrupting one or more of the β-mannosyltransferase genes (e.g., BMT1, BMT2, BMT3, and BMT4) (See, U.S. Pat. No. 7,465,577 and U.S. Pat. No. 7,713,719). The deletion or disruption of BMT2 and one or more of BMT1, BMT3, and BMT4 also reduces or eliminates detectable cross reactivity to antibodies against host cell protein.

Yield of glycoprotein can in some situations be improved by overexpressing nucleic acid molecules encoding mammalian or human chaperone proteins or replacing the genes encoding one or more endogenous chaperone proteins with nucleic acid molecules encoding one or more mammalian or human chaperone proteins. In addition, the expression of mammalian or human chaperone proteins in the host cell also appears to control O-glycosylation in the cell. Thus, further included are the host cells herein wherein the function of at least one endogenous gene encoding a chaperone protein has been reduced or eliminated, and a vector encoding at least one mammalian or human homolog of the chaperone protein is expressed in the host cell. Also included are host cells in which the endogenous host cell chaperones and the mammalian or human chaperone proteins are expressed. In further aspects, the lower eukaryotic host cell is a yeast or filamentous fungi host cell. Examples of the use of chaperones of host cells in which human chaperone proteins are introduced to improve the yield and reduce or control O-glycosylation of recombinant proteins has been disclosed in Published International Application No. WO 2009105357 and WO2010019487 (the disclosures of which are incorporated herein by reference). Like above, further included are lower eukaryotic host cells wherein, in addition to replacing the genes encoding one or more of the endogenous chaperone proteins with nucleic acid molecules encoding one or more mammalian or human chaperone proteins or overexpressing one or more mammalian or human chaperone proteins as described above, the function or expression of at least one endogenous gene encoding a protein O-mannosyltransferase (PMT) protein is reduced, disrupted, or deleted. In particular embodiments, the function of at least one endogenous PMT gene selected from the group consisting of the PMT1, PMT2, PMT3, and PMT4 genes is reduced, disrupted, or deleted.

In addition, O-glycosylation may have an effect on an antibody or Fab fragment's affinity and/or avidity for an antigen. This can be particularly significant when the ultimate host cell for production of the antibody or Fab is not the same as the host cell that was used for selecting the antibody. For example, O-glycosylation might interfere with an antibody's or Fab fragment's affinity for an antigen, thus an antibody or Fab fragment that might otherwise have high affinity for an antigen might not be identified because O-glycosylation may interfere with the ability of the antibody or Fab fragment to bind the antigen. In other cases, an antibody or Fab fragment that has high avidity for an antigen might not be identified because O-glycosylation interferes with the antibody's or Fab fragment's avidity for the antigen. In the preceding two cases, an antibody or Fab fragment that might be particularly effective when produced in a mammalian cell line might not be identified because the host cells for identifying and selecting the antibody or Fab fragment was of another cell type, for example, a yeast or fungal cell (e.g., a Pichia pastoris host cell). It is well known that O-glycosylation in yeast can be significantly different from O-glycosylation in mammalian cells. This is particularly relevant when comparing wild type yeast O-glycosylation with mucin-type or dystroglycan type O-glycosylation in mammals. In particular cases, O-glycosylation might enhance the antibody or Fab fragments affinity or avidity for an antigen instead of interfere with antigen binding. This effect is undesirable when the production host cell is to be different from the host cell used to identify and select the antibody or Fab fragment (for example, identification and selection is done in yeast and the production host is a mammalian cell) because in the production host the O-glycosylation will no longer be of the type that caused the enhanced affinity or avidity for the antigen. Therefore, controlling O-glycosylation can enable use of the materials and methods herein to identify and select antibodies or Fab fragments with specificity for a particular antigen based upon affinity or avidity of the antibody or Fab fragment for the antigen without identification and selection of the antibody or Fab fragment being influenced by the O-glycosylation system of the host cell. Thus, controlling O-glycosylation further enhances the usefulness of yeast or fungal host cells to identify and select antibodies or Fab fragments that will ultimately be produced in a mammalian cell line.

Those of ordinary skill in the art would further appreciate and understand how to utilize the methods and materials described herein in combination with other Pichia pastoris and yeast cell lines that have been genetically engineered to produce specific N-glycans or sialylated glycoproteins, such as, but, not limited to, the host organisms and cell lines described above that have been genetically engineered to produce specific galactosylated or sialylated forms. See, for example, U.S. Publication No. 2006-0286637, Production of Sialylated N-Glycans in Lower Eukaryotes, in which the pathway for galactose uptake and utilization as a carbon source has been genetically modified, the description of which is incorporated herein as if set forth at length.

Additionally, the methods herein can be used to produce the above described recombinant Fc-containing polypeptides in other lower eukaryotic cell lines that do not have α-2,3 sialyltransferase activity but which have been engineered to produce human-like and human glycoproteins comprising α-2,3-sialyltransferase activity. The methods can also be used to produce the above described recombinant Fc-containing polypeptides in eukaryotic cell lines in which production of sialylated N-glycans is an innate feature.

Levels of α-2,3 and α-2,6 linked sialic acid on the Fc-containing polypeptides can be measured using well known techniques including nuclear magnetic resonance (NMR), normal phase high performance liquid chromatography (HPLC), and high performance anion exchange chromatography with pulsed amperometric detection (HPAEC-PAD).

### Production of Fc-Containing Polypeptides

The Fc-containing polypeptides of the invention can be made according to any method known in the art suitable for generating polypeptides comprising an Fc region having sialylated N-glycans. In one embodiment, the Fc-containing polypeptide is an antibody or an antibody fragment (including, without limitation a polypeptide consisting of or consisting essentially of the Fc region of an antibody). In another embodiment, the Fc-containing polypeptide is an immunoadhesin. Methods of preparing antibody, antibody fragments and immunoadhesins are well known in the art. Methods of introducing point mutations into a polypeptide, for example site directed mutagenesis, are also well known in the art.

In one embodiment, the Fc-containing polypeptides of the invention are expressed in a host cell that has naturally expresses an α-2,3 sialic acid transferase. In one embodiment, the Fc-containing polypeptides of the invention are expressed in a host cell that has been transformed with a nucleic acid encoding an α-2,3 sialic acid transferase. In one embodiment the host cell is a mammalian cell. In one embodiment, the host cell is a lower eukaryotic host cell. In one embodiment, the host cell is fungal host cell. In one embodiment, the host cell is Pichia sp. In one embodiment, the host cell is Pichia pastoris. In one embodiment, said host cell is capable of producing Fc-polypeptides comprising sialylated N-glycans, wherein the sialic acid residues in the sialylated N-glycans contain alpha-2,3 linkages. In one embodiment, said host cell is capable of producing Fc-containing polypeptides, wherein at least 30%, 40%, 50%, 60%, 70%, 80% or 90% of the N-glycans on the Fc-containing polypeptide comprise an N-linked oligosaccharide structure selected from the group consisting of SA₂Gal₍₁₋₄₎GlcNAc₍₂₋₄₎Man₃GlcNAc₂. In one embodiment, at least 30%, 40%, 50%, 60%, 70%, 80% or 90% of the N-glycans on the Fc-containing polypeptide comprise an N-linked oligosaccharide structure selected from the group consisting of SA₂Gal₂GlcNAc₂Man₃GlcNAc₂. In one embodiment, at least 80% of the N-glycans on the Fc-containing polypeptide comprise an N-linked oligosaccharide structure selected from the group consisting of SA₂Gal₂GlcNAc₂Man₃GlcNAc₂. In any of the above embodiments, the SA could be NANA or NGNA, or an analog or derivative of NANA or NGNA. In one embodiment, at least 30%, 40%, 50%, 60%, 70%, 80% or 90% of the N-glycans on the Fc-containing polypeptide comprise an N-linked oligosaccharide structure selected from the group consisting of NANA₂Gal₂GlcNAc₂Man₃GlcNAc. In one embodiment, the sialic acid residues in the sialylatd N-glycans are attached exclusively via α-2,3 linkages.

### N-Glycan Analysis of Fc Containing Polypeptides

The N-glycan composition of the antibodies produced herein in glycoengineered Pichia pastoris GFI5.0 and GFI6.0 strains can be analyzed by matrix-assisted laser desorption ionization/time-of-flight (MALDI-TOF) mass spectrometry after release from the antibody with peptide-N-glycosidase F. Released carbohydrate composition can be quantitated by HPLC on an Allentech Prevail carbo (Alltech Associates, Deerfield Ill.) column.

### Methods of Activating Immune Cells

The invention also comprises a method of activating immune cells or enhancing the effector function of immune cells by contacting an immune cell with an Fc-containing polypeptide comprising α-2,3-linked sialic acid.

The invention also comprises a method of activating immune cells or enhancing the effector function of immune cells by contacting an immune cell with an Fc-containing polypeptide comprising an increased amount of α-2,3-linked sialic acid compared to the amount of α-2,3-linked in a parent polypeptide. In one embodiment, the Fc-containing polypeptide has one or more of the following properties when compared to the parent Fc-containing polypeptide: (a) increased effector function; (b) increased ability to recruit immune cells (such as T cells, B cells, and/or effector cells/macrophages); and (c) increased inflammatory properties. In one embodiment, an Fc-containing polypeptide having increased inflammatory properties is an Fc-containing polypeptide which has increased/enhanced ability to stimulate the secretion of factors/cytokines which cause inflammation, for example, IL-1, IL-6, RANKL and TNF.

In some embodiments of the invention, the amount of α-2,3-linked sialic acid is increased by expressing the Fc-containing polypeptide in a host cell that has been transformed with a nucleic acid encoding an α-2,3 sialyltransferase. In one embodiment, the host cell is a yeast cell. In some embodiments, the amount of α-2,3-linked sialic acid is further increased by producing the Fc-containing polypeptide under cell culture conditions which result in increased sialic acid content. In another embodiment, the amount of α-2,3-linked sialic acid is increased by introducing one or more mutations in the Fc region of the Fc-containing polypeptide. In one embodiment, the mutations are introduced at one or more locations selected from the group consisting of: 241, 243, 264, 265, 267, 296, 301 and 328, wherein the numbering is according to the EU index as in Kabat. In one embodiment, the mutations are introduced at two or more locations selected from the group consisting of: 241, 243, 264, 265, 267, 296, 301 and 328. In one embodiment, the mutations are introduced at positions 243 and 264 of the Fc region. In one embodiment, the mutations at positions 243 and 264 are selected from the group consisting of: F243A and V264A; F243Y and V264G; F243T and V264G; F243L and V264A; F243L and V264N; and F243V and V264G. In one embodiment, the mutations introduced are F243A and V264A. In another embodiment, the mutations introduced are: F243A, V264A, S267E, and L328F.

The above described methods of activating immune cells could be used to treat cancer or infectious diseases (such as chronic viral infenctions) or could be used as an adjuvant to a prophylactic or therapeutic vaccine.

In some embodiments of the above described methods, all of the sialic acid residues in the Fc-containing polypeptide are attached exclusively via an α-2,3 linkage. In other embodiments, most of the sialic acid residues in the Fc-containing polypeptide are attached via an α-2,3 linkage. In other embodiments, some of the sialic acid residues in the Fc-containing polypeptide are attached via an α-2,3 linkage while others are attached via an α-2,6 linkage.

In some embodiments of the above described methods, at least 30%, 40%, 50%, 60%, 70%, 80% or 90% of the N-glycans on the Fc-containing polypeptide comprise an oligosaccharide structure selected from the group consisting of SA₍₁₋₄₎Gal₍₁₋₄₎GlcNAc₍₂₋₄₎Man₃GlcNA₂.

In some embodiments of the above described methods, at least 30%, 40%, 50%, 60%, 70%, 80% or 90% of the N-glycans on the Fc-containing polypeptide comprise an oligosaccharide structure selected from the group consisting of SA₂Gal₍₁₋₄₎GlcNAc₍₂₋₄₎Man₃GlcNAc₂.

In some embodiments of the above described methods, at least 30%, 40%, 50%, 60%, 70%, 80% or 90% of the N-glycans on the Fc-containing polypeptide comprise an oligosaccharide structure consisting of SA₂Gal₂GlcNAc₂Man₃GlcNAc₂. In one embodiment, at least 80% of the N-glycans on the Fc-containing polypeptide comprise an N-linked oligosaccharide structure selected from the group consisting of SA₂Gal₂GlcNAc₂Man₃ GlcNAc₂.

In some embodiments of the above described methods, at least 30%, 40%, 50%, 60%, 70%, 80% or 90% of the N-glycans on the Fc-containing polypeptide comprise an oligosaccharide structure selected from the group consisting of NANA₂Gal₍₁₋₄₎GlcNAc₍₂₋₄₎Man₃GlcNAc₂.

In some embodiments of the above described methods, at least 30%, 40%, 50%, 60%, 70%, 80% or 90% of the N-glycans on the Fc-containing polypeptide comprise an oligosaccharide structure consisting of NANA₂Gal₂GlcNAc₂Man₃GlcNAc₂. In one embodiment, at least 80% of the N-glycans on the Fc-containing polypeptide comprise an N-linked oligosaccharide structure selected from the group consisting of NANA₂Gal₂GlcNAc₂Man₃GlcNAc₂.

In some embodiments, the Fc containing polypeptide comprises the amino acid sequence of SEQ ID NO:6 or SEQ ID NO:7, plus one or more mutations which result in an increased amount of sialic acid. In another embodiments, the Fc containing polypeptide comprises the amino acid sequence of SEQ ID NO:6 or SEQ ID NO:7, plus one, two, three or four mutations which result in an increased amount of sialic acid (for example, mutations at one or more locations selected from the group consisting of: 241, 243, 264, 265, 267, 296, 301 and 328, wherein the numbering is according to the EU index as in Kabat). In one embodiment, the Fc-containing polypeptide comprises the amino acid sequence of SEQ ID NO: 8 or 9.

In another embodiment, the amount of α-2,3-linked sialic acid is increased by expressing the Fc-containing polypeptide in a host cell that has been transformed with a nucleic acid encoding an α-2,3 sialic acid transferase and by introducing one or more mutations in the Fc region of the Fc-containing polypeptide. In one embodiment the host cell is a yeast cell. The mutation could be any of the Fc mutations described above.

The invention also comprises a method of increasing an immune response to an antigen, comprising: contacting an immune cell with: (i) an antigen and (ii) an Fc-containing polypeptide comprising α-2,3-linked sialic acid, such that an immune response to the antigen is increased or enhanced. This method could be conducted in vivo (in a subject) or ex vivo. In one embodiment, the invention comprises: (i) obtaining immune cells from a patient, (ii) contacting the immune cells with an Fc-containing polypeptide comprising α-2,3 linked sialic acid, and (iii) then administering the immune cells to the patient. In one embodiment, the Fc-containing polypeptide comprises an increased amount of α-2,3-linked sialic acid compared to the amount of α-2,3-linked in a parent polypeptide.

### Methods of Treatment

The Fc-containing polypeptides of the invention could be used in the treatment of diseases or disorders where destruction or elimination of tissue or foreign microorganisms is desired. For example, the Fc-containing polypeptides of the invention could be used to treat neoplastic diseases or infectious (e.g., bacterial, viral, fungal or yeast) diseases. Further, the Fc-containing polypeptides of the invention could be used as vaccine adjuvants.

The invention comprises a method of enhancing an immune response in a subject in need thereof comprising: administering to the subject a therapeutically effective amount of an Fc-containing polypeptide comprising α-2,3-linked sialic acid. In one embodiment, the subject as an infectious disease. In another embodiment, the subject has a neoplastic disease.

The invention comprises a method of enhancing an immune response in a subject in need thereof comprising: administering to the subject a therapeutically effective amount of an Fc-containing polypeptide comprising an increased amount of α-2,3-linked sialic acid compared to the amount of α-2,3-linked in a parent polypeptide. In one embodiment, the subject as an infectious disease. In another embodiment, the subject has a neoplastic disease. In some embodiments, the amount of α-2,3-linked sialic acid is increased by expressing the Fc-containing polypeptide in a host cell that has been transformed with a nucleic acid encoding an α-2,3 sialic acid transferase. In one embodiment, the host cell is a yeast cell. In some embodiments, the amount of α-2,3-linked sialic acid is further increased by producing the Fc-containing polypeptide under cell culture conditions which result in increased sialic acid content. In another embodiment, the amount of α-2,3-linked sialic acid is increased by introducing one or more mutations in the Fc region of the Fc-containing polypeptide. In one embodiment, the mutations are introduced at one or more locations selected from the group consisting of: 241, 243, 264, 265, 267, 296, 301 and 328, wherein the numbering is according to the EU index as in Kabat. In one embodiment, the mutations are introduced at two or more locations selected from the group consisting of: 241, 243, 264, 265, 267, 296, 301 and 328. In one embodiment, the mutations are introduced at positions 243 and 264 of the Fc region. In one embodiment, the mutations at positions 243 and 264 are selected from the group consisting of F243A and V264A; F243Y and V264G; F243T and V264G; F243L and V264A; F243L and V264N; and F243V and V264G. In one embodiment, the mutations introduced are F243A and V264A. In another embodiment, the mutations introduced are: F243A, V264A, S267E, and L328F. In another embodiment, the amount of of α-2,3-linked sialic acid is increased by expressing the Fc-containing polypeptide in a host cell that has been transformed with a nucleic acid encoding an α-2,3 sialic acid transferase and by introducing one or more mutations in the Fc region of the Fc-containing polypeptide. The mutation could be any of the Fc mutations described herein.

In some embodiments of the above described methods of treatment, all of the sialic acid residues in the Fc-containing polypeptide are attached exclusively via an α-2,3 linkage. In other embodiments, most of the sialic acid residues in the Fc-containing polypeptide are attached via an α-2,3 linkage. In other embodiments, some of the sialic acid residues in the Fc-containing polypeptide are attached via an α-2,3 linkage while others are attached via an α-2,6 linkage.

In some embodiments, at least 30%, 40%, 50%, 60%, 70% of the N-glycans on the Fc-containing polypeptide comprise an oligosaccharide structure selected from the group consisting of SA₍₁₋₄₎Gal₍₁₋₄₎GlcNAc₍₂₋₄₎Man₃GlcNAc₂. In some embodiments, at least 30%, 40%, 50%, 60%, 70% of the N-glycans on the Fc-containing polypeptide comprise an oligosaccharide structure consisting of SA₂Gal₂GlcNAc₂Man₃GlcNAc₂. In one embodiment, at least 80% of the N-glycans on the Fc-containing polypeptide comprise an N-linked oligosaccharide structure selected from the group consisting of SA₂Gal₂GlcNAc₂Man₃GlcNAc₂. In some embodiments, at least 30%, 40%, 50%, 60%, 70% of the N-glycans on the Fc-containing polypeptide comprise an oligosaccharide structure consisting of NANA₂Gal₂GlcNAc₂Man₃GlcNAc₂. In one embodiment, at least 80% of the N-glycans on the Fc-containing polypeptide comprise an N-linked oligosaccharide structure selected from the group consisting of NANA₂Gal₂GlcNAc₂Man₃GlcNAc₂.

In one embodiment, the Fc containing polypeptide comprises the amino acid sequence of SEQ ID NO: 6 or SEQ ID NO:7. In one embodiment, the Fc containing polypeptide comprises the amino acid sequence of SEQ ID NO:6 or SEQ ID NO:7, plus one or more mutations which result in an increased amount of sialic acid. In one embodiment, the Fc containing polypeptide comprises the amino acid sequence of SEQ ID NO:6 or SEQ ID NO:7, plus one, two, three or four mutations which result in an increased amount of sialic acid (for example, mutations at one or more locations selected from the group consisting of: 241, 243, 264, 265, 267, 296, 301 and 328, wherein the numbering is according to the EU index as in Kabat). In some embodiment, the mutations are: F243A/V264A; F243Y/V264G; F243T/V264G; F243L/V264A; F243L/V264N; F243V/V264G; F243A/V264A/S267E/L328F.

In one embodiment, the Fc containing polypeptide comprises the amino acid sequence of SEQ ID NO:8 or SEQ ID NO:9.

In some embodiments of the above described methods, the Fc-containing polypeptide has one or more of the following properties when compared to a parent Fc-containing polypeptide: (a) increased effector function; (b) increased ability to recruit immune cells (such as T cells, B cells, and or effector cells/macrophages); and (c) increased inflammatory properties.

In one embodiment, the invention comprises a method of enhancing an immune response in a subject in need thereof comprising administering to the subject a therapeutically effective amount of an Fc-containing polypeptide comprising sialylated N-glycans, wherein the sialic acid residues in the sialylated N-glycans contain α-2,3 linkages, and wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃GlcNAc₂. In one embodiment, the subject has, or is at risk of developing, an infectious disease or a neoplastic disease. In one embodiment, at least 30%, 40%, 50%, 60%, 70%, 80% or 90% of the N-glycans on the Fc-containing polypeptide comprise an N-linked oligosaccharide structure selected from the group consisting of SA₂Gal₍₁₋₄₎GlcNAc₍₂₋₄₎Man₃GlcNAc₂. In one embodiment, at least 30%, 40%, 50%, 60%, 70%, 80% or 90% of the N-glycans on the Fc-containing polypeptide comprise an N-linked oligosaccharide structure consisting of SA₂Gal₂GlcNAc₂Man₃GlcNAc₂. In one embodiment, at least 80% of the N-glycans on the Fc-containing polypeptide comprise an N-linked oligosaccharide structure selected from the group consisting of SA₂Gal₂GlcNAc₂Man₃GlcNAc₂. In one embodiment, at least 30%, 40%, 50%, 60%, 70%, 80% or 90% of the N-glycans on the Fc-containing polypeptide comprise an N-linked oligosaccharide structure consisting of NANA₂Gal₂GlcNAc₂Man₃GlcNAc₂. In one embodiment, at least 80% of the N-glycans on the Fc-containing polypeptide comprise an N-linked oligosaccharide structure selected from the group consisting of NANA₂Gal₂GlcNAc₂Man₃GlcNAc₂. In one embodiment, the Fc polypeptide comprises N-glycans at a position that corresponds to the Asn297 site of a full-length heavy chain antibody, wherein the numbering is according to the EU index as in Kabat. In one embodiment, the N-glycans lack fucose. In another embodiment, the N-glycans further comprise a core fucose. In one embodiment, all of the sialic acid residues in the Fc-containing polypeptide are attached exclusively via an α-2,3 linkage.

In one embodiment, the invention comprises a method of enhancing an immune response in a subject in need thereof comprising administering to the subject a therapeutically effective amount of an Fc-containing polypeptide comprising sialylated N-glycans, wherein the sialic acid residues in the sialylated N-glycans contain α-2,3 linkages, and wherein at least 30%, 40%, 50%, 60%, 70%, 80% or 90% of the N-glycans on the Fc-containing polypeptide comprise an N-linked oligosaccharide structure selected from the group consisting of SA(1-4)Gal(1-4)GlcNAc(1-4)Man(>=3)GlcNAc2. In one embodiment at least 30%, 40%, 50%, 60%, 70%, 80% or 90% of the N-glycans on the Fc-containing polypeptide comprise an oligosaccharide structure selected from the group consisting of SA(1-3)Gal(1-3)GlcNAc(1-3)Man3GlcNAc2. In one embodiment, the sialic acid residues in the sialylatd N-glycans are attached exclusively via α-2,3 linkages. In one embodiment, the Fc polypeptide comprises N-glycans at a position that corresponds to the Asn297 site of a full-length heavy chain antibody, wherein the numbering is according to the EU index as in Kabat. In one embodiment, the N-glycans lack fucose. In another embodiment, the N-glycans further comprise a core fucose.

In one embodiment, the invention comprises a method of enhancing an immune response in a subject in need thereof comprising administering to the subject a therapeutically effective amount of an Fc-containing polypeptide comprising sialylated N-glycans, wherein the sialic acid residues in the sialylated N-glycans contain α-2,3 linkages, and wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃GlcNAc₂. In one embodiment, all of the sialic acid residues in the Fc-containing polypeptide are attached exclusively via an α-2,3 linkage. In one embodiment, the N-glycans lack fucose. In another embodiment, the N-glycans further comprise a core fucose. In one embodiment, the Fc polypeptide is an antibody or antibody fragment comprising sialylated N-glycans. In one embodiment, the Fc polypeptide comprises N-glycans at a position that corresponds to the Asn297 site of a full-length heavy chain antibody, wherein the numbering is according to the EU index as in Kabat. In one embodiment, the Fc polypeptide is an antibody or antibody fragment comprising or consisting essentially of SEQ ID NO:6 or SEQ ID NO:7. In one embodiment the Fc-containing polypeptide comprises or consists of the amino acid sequence of SEQ ID NO: 6 or SEQ ID NO: 7, plus one or more mutations which result in an increased amount of sialic acid when compared to the amount of sialic acid in a parent polypeptide. In one embodiment the Fc-containing polypeptide comprises or consists of the amino acid sequence of SEQ ID NO: 6 or SEQ ID NO: 7, plus one, two, three or four mutations which result in an increased amount of sialic acid when compared to the amount of sialic acid in a parent polypeptide. In one embodiment, the parent polypeptide comprises the amino acid sequence of SEQ ID NO:6 or SEQ ID NO:7. In one embodiment, the Fc-containing polypeptide is an antibody or antibody fragment comprising mutations at positions 243 and 264 of the Fc region wherein the numbering is according to EU index as in Kabat. In one embodiment, the mutations are F243A and V264A.

In one embodiment, the Fc-containing polypeptide of the invention will be administered a dose of between 1 to 100 milligrams per kilograms of body weight. In one embodiment, the Fc-containing polypeptide of the invention will be administered a dose of between 0.001 to 10 milligrams per kilograms of body weight. In one embodiment, the Fc-containing polypeptide of the invention will be administered a dose of between 0.001 to 0.1 milligrams per kilograms of body weight. In one embodiment, the Fc-containing polypeptide of the invention will be administered a dose of between 0.001 to 0.01 milligrams per kilograms of body weight.

The invention comprises a method of boosting immunogenicity during vaccination (either prophylactic or therapeutic) comprising: administering to the subject a therapeutically effective amount of an Fc-containing polypeptide comprising α-2,3-linked sialic acid. In one embodiment, the Fc-containing polypeptide is an antibody or immunoadhesin that recognizes a viral or bacterial antigen. In one embodiment, the Fc-containing polypeptide comprises an an increased amount of α-2,3-linked sialic acid compared to the amount of α-2,3-linked in a parent polypeptide. The amount of sialic acid in an Fc-containing polypeptide can be increased using any of the method, including the methods disclosed above.

The invention comprises a method of boosting immunogenicity during vaccination (either prophylactic or therapeutic) comprising: administering to the subject a therapeutically effective amount of an Fc-containing polypeptide comprising sialylated N-glycans, wherein the sialic acid residues in the sialylated N-glycans contain α-2,3 linkages, and wherein at least 30%, 40%, 50%, 60%, 70%, 80% or 90% of the N-glycans on the Fc-containing polypeptide comprise an N-linked oligosaccharide structure selected from the group consisting of SA(1-4)Gal(1-4)GlcNAc(1-4)Man(>=3)GlcNAc2. In one embodiment at least 30%, 40%, 50%, 60%, 70%, 80% or 90% of the N-glycans on the Fc-containing polypeptide comprise an oligosaccharide structure selected from the group consisting of SA(1-3)Gal(1-3)GlcNAc(1-3)Man3GlcNAc2. In one embodiment, all of the sialic acid residues in the Fc-containing polypeptide are attached exclusively via an α-2,3 linkage. In one embodiment, the Fc polypeptide comprises N-glycans at a position that corresponds to the Asn297 site of a full-length heavy chain antibody, wherein the numbering is according to the EU index as in Kabat. In one embodiment, the N-glycans lack fucose. In another embodiment, the N-glycans further comprise a core fucose. In one embodiment, the Fc-containing polypeptide binds a viral or bacterial antigen.

The invention also comprises the use of an Fc-containing polypeptide comprising α-2,3-linked sialic acid as a vaccine adjuvant. In one embodiment, at least 30%, 40%, 50%, 60%, 70% of the N-glycans on the Fc-containing polypeptide comprise an oligosaccharide structure selected from the group consisting of SA₍₁₋₄₎Gal₍₁₋₄₎GlcNAc₍₂₋₄₎Man₃GlcNAc₂.

The invention also comprises the use of an Fc-containing polypeptide a vaccine adjuvant. In one embodiment, the Fc-containing polypeptide comprises sialylated N-glycans, wherein the sialic acid residues in the sialylated N-glycans contain α-2,3 linkages, and wherein at least 30%, 40%, 50%, 60%, 70%, 80% or 90% of the N-glycans on the Fc-containing polypeptide comprise an N-linked oligosaccharide structure selected from the group consisting of SA(1-4)Gal(1-4)GlcNAc(1-4)Man(>=3)GlcNAc2. In one embodiment at least 30%, 40%, 50%, 60%, 70%, 80% or 90% of the N-glycans on the Fc-containing polypeptide comprise an oligosaccharide structure selected from the group consisting of SA(1-3)Gal(1-3)GlcNAc(1-3)Man3GlcNAc2. In one embodiment, all of the sialic acid residues in the Fc-containing polypeptide are attached exclusively via an α-2,3 linkage. In one embodiment, the N-glycans lack fucose. In another embodiment, the N-glycans further comprise a core fucose. In one embodiment, the Fc polypeptide comprises N-glycans at a position that corresponds to the Asn297 site of a full-length heavy chain antibody, wherein the numbering is according to the EU index as in Kabat. In one embodiment, the Fc-containing polypeptide binds a viral or bacterial antigen.

In some embodiments, the Fc-containing polypeptide of the invention may be combined with a second therapeutic agent or treatment modality. In some embodiments, the Fc-containing polypeptide of the invention (comprising α-2,3-linked sialic acid) may be combined with another therapeutic antibody useful for the treatment of cancer or infectious disease.

In some embodiments, the Fc-containing polypeptide of the invention (comprising α-2,3-linked sialic acid) is combined with a vaccine to prevent or treat cancer or infectious disease. As a non-limiting example, the Fc-containing polypeptide of the invention (comprising α-2,3-linked sialic acid) is combined with a protein, peptide or DNA vaccine containing one or more antigens which are relevant to the cancer or infection to be treated, or a vaccine comprising of dendritic cells pulsed with such an antigen. Another embodiment includes the use of the Fc-containing polypeptide of the invention (comprising α-2,3-linked sialic acid) with (attenuated) cancer cell or whole virus vaccines.

### Methods of Increasing the Effector Function of an Fc-Containing Polypeptide

The invention also comprises a method of increasing the effector function or inflammatory properties of an Fc containing polypeptide: (i) selecting a parent Fc-containing polypeptide and (ii) adding or increasing the amount of, α-2,3-linked sialic acid (for example SA₍₁₋₄₎Gal₍₁₋₄₎GlcNAc₍₂₋₄₎Man₃GlcNAc₂, wherein the sialic acid residues are exclusively attached to galactose through an α-2,3 linkage) in the parent Fc-containing polypeptide. In one embodiment, the parent Fc containing polypeptide is a polypeptide that is useful in treating an infectious disease or a neoplastic disease, or that can be used as a vaccine adjuvant.

The invention also comprising a method of increasing the anti-tumor potency of an Fc-containing polypeptide comprising: (i) selecting a parent Fc-containing polypeptide and (ii) adding or increasing the amount of α-2,3-linked sialic acid (for example SA₍₁₋₄₎Gal₍₁₋₄₎GlcNAc₍₂₋₄₎Man₃GlcNAc₂), wherein the sialic acid residues are exclusively attached to galactose through an α-2,3 linkage in the parent Fc-containing polypeptide.

The invention also comprising a method of increasing the anti-tumor potency of an Fc-containing polypeptide comprising: (i) selecting a parent Fc-containing polypeptide and (ii) expressing said Fc-containing polypeptide in a host cell that has been transformed with a nucleic acid encoding an α-2,3 sialic acid transferase. In one embodiment the host cell is a mammalian cell. In one embodiment, the host cell is a lower eukaryotic host cell. In one embodiment, the host cell is fungal host cell. In one embodiment, the host cell is Pichia sp. In one embodiment, the host cell is Pichia pastoris. In one embodiment, said host cell is capable of producing Fc-polypeptides comprising sialylated N-glycans, wherein the sialic acid residues in the sialylated N-glycans contain alpha-2,3 linkages. In one embodiment, said host cell is capable of producing Fc-containing polypeptides, wherein at least 30%, 40%, 50%, 60%, 70%, 80% or 90% of the N-glycans on the Fc-containing polypeptide comprise an N-linked oligosaccharide structure selected from the group consisting of SA₂Gal₍₁₋₄₎GlcNAc₍₂₋₄₎Man₃GlcNAc₂. In one embodiment, at least 30%, 40%, 50%, 60%, 70%, 80% or 90% of the N-glycans on the Fc-containing polypeptide comprise an N-linked oligosaccharide structure consisting of SA₂Gal₂GlcNAc₂Man₃GlcNAc₂. In one embodiment, at least 80% of the N-glycans on the Fc-containing polypeptide comprise an N-linked oligosaccharide structure selected from the group consisting of SA₂Gal₂GlcNAc₂Man₃GlcNAc₂. In any of the above embodiments, the SA could be NANA or NGNA, or an analog or derivative of NANA or NGNA. In one embodiment, at least 30%, 40%, 50%, 60%, 70%, 80% or 90% of the N-glycans on the Fc-containing polypeptide comprise an N-linked oligosaccharide structure consisting of NANA₂Gal₂GlcNAc₂Man₃GlcNAc. In one embodiment, all of the sialic acid residues in the Fc-containing polypeptide are attached exclusively via an α-2,3 linkage. In one embodiment, the N-glycans lack fucose. In another embodiment, the N-glycans further comprise a core fucose.

### Biological Targets

It should be noted that while, in the examples that follow, Applicants exemplifiy the materials and methods of the invention using IgG1 antibodies having sequences similar to those for commercially available anti-TNF antibodies, the invention is not limited to the disclosed antibodies. Those of ordinary skill in the art would recognize and appreciate that the materials and methods herein could be used to produce any Fc-containing polypeptide, or bioactive form thereof, for which the characteristics of enhanced effector function cells would be desirable. It should further be noted that there is no restriction as to the type of Fc-containing polypeptide or antibody so produced by the invention. The Fc region of the Fc-containing polypeptide could be from an IgA, IgD, IgE, IgG or IgM. In one embodiment, the Fc region of the Fc-containing polypeptide is from an IgG, including IgG1, IgG2, IgG3 or IgG4. In one embodiment, the Fc region of the Fc-containing polypeptide is from an IgG1. In one embodiment, the Fc region of the Fc-containing polypeptide is from an IgG1. In specific embodiments, antibodies or antibody fragments produced by the materials and methods herein can be humanized, chimeric or human antibodies.

In some embodiments, the Fc-containing polypeptides of the invention will bind to a biological target that is involved in neoplastic disease (i.e., cancer).

In some embodiments, the Fc-containing polypeptide of the invention will bind to an antigen selected from HER2, HERS, EGF, EGFR, VEGF, VEGFR, IGFR, PD-1, PD-1L, BTLA, CTLA-4, GITR, mTOR, CS1, CD20, CD22, CD27, CD28, CD30, CD33, CD40, CD52, CD137, CAl25, MUC1, PEM antigen, Ep-CAM, 17-1a, CEA, AFP, HLA-DR, GD2-ganglioside, SK-1 antigen, Lag3, Tim3, CTLA4, TIGIT, SIRPa, ICOS, Trem12, NCR3, HVEM, OX40 and 4-1BB.

In other embodiments, the Fc-containing polypeptide of the invention will bind to any pathogenic antigen (for example, a viral or bacterial antigen). In some embodiments, the Fc-containing polypeptide of the invention will bind to gp120, gp41, Flu HA, an HBV antigen, or an HCV antigen.

### Pharmaceutical Formulations

The invention also comprises pharmaceutical formulations comprising an Fc-containing polypeptide comprising sialylated N-glycans, wherein the sialic acid residues in the sialylated N-glycans contain α-2,3 linkages, and a pharmaceutically acceptable carrier. In one embodiment, all of the silaic acid residues in the sialylated N-glycans are attached exclusively via α-2,3 linkages. In one embodiment, the Fc-containing polypeptide is an antibody or an antibody fragment or an immunoadhesin.

In one embodiment, the invention relates a pharmaceutical composition comprising an Fc-containing polypeptide, wherein at least 30%, 40%, 50%, 60%, 70%, 80% or 90% of the N-glycans on the Fc-containing polypeptide comprise an oligosaccharide structure selected from the group consisting of SA₍₁₋₄₎Gal₍₁₋₄₎GlcNAc₍₂₋₄₎Man₃GlcNAc₂, wherein the sialic acid residues are exclusively attached through an α-2,3 linkage. In one embodiment, at least 30%, 40%, 50%, 60%, 70%, 80% or 90% of the N-glycans on the Fc-containing polypeptide comprise an oligosaccharide structure consisting of SA₂Gal₂GlcNAc₂Man₃GlcNAc₂. In one embodiment, at least 80% of the N-glycans on the Fc-containing polypeptide comprise an N-linked oligosaccharide structure selected from the group consisting of SA₂Gal₂GlcNAc₂Man₃GlcNAc₂. In one embodiment, at least 30%, 40%, 50%, 60%, 70%, 80% or 90% of the N-glycans on the Fc-containing polypeptide comprise an oligosaccharide structure consisting of NANA₂Gal₂GlcNAc₂Man₃GlcNAc₂. In one embodiment, at least 80% of the N-glycans on the Fc-containing polypeptide comprise an N-linked oligosaccharide structure selected from the group consisting of NANA₂Gal₂GlcNAc₂Man₃GlcNAc₂. In one embodiment, the N-glycans lack fucose. In another embodiment, the N-glycans further comprise a core fucose.

In one embodiment, the invention comprises a pharmaceutical formulation comprising an Fc-containing polypeptide, wherein the Fc-containing polypeptide comprises sialylated N-glycans, wherein the sialic acid residues in the sialylated N-glycans contain α-2,3 linkages, and wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃GlcNAc₂. In one embodiment, at least 30%, 40%, 50%, 60%, 70%, 80% or 90% of the N-glycans on the Fc-containing polypeptide comprise an N-linked oligosaccharide structure selected from the group consisting of SA₂Gal₍₁₋₄₎GlcNAc₍₂₋₄₎Man₃GlcNAc₂. In one embodiment, at least 30%, 40%, 50%, 60%, 70%, 80% or 90% of the N-glycans on the Fc-containing polypeptide comprise an N-linked oligosaccharide structure consisting of SA₂Gal₂GlcNAc₂Man₃GlcNAc₂. In one embodiment, at least 80% of the N-glycans on the Fc-containing polypeptide comprise an N-linked oligosaccharide structure selected from the group consisting of SA₂Gal₂GlcNAc₂Man₃GlcNAc₂. In one embodiment, at least 30%, 40%, 50%, 60%, 70%, 80% or 90% of the N-glycans on the Fc-containing polypeptide comprise an N-linked oligosaccharide structure consisting of NANA₂Gal₂GlcNAc₂Man₃GlcNAc₂. In one embodiment, at least 80% of the N-glycans on the Fc-containing polypeptide comprise an N-linked oligosaccharide structure selected from the group consisting of NANA₂Gal₂GlcNAc₂Man₃GlcNAc₂. In one embodiment, all of the silaic acid residues in the sialylated N-glycans are attached exclusively via α-2,3 linkages. In one embodiment, the N-glycans lack fucose. In another embodiment, the N-glycans further comprise a core fucose. In one embodiment, the N-glycans are attached at a position that corresponds to the Asn297 site of a full-length heavy chain antibody, wherein the numbering is according to the EU index as in Kabat.

In one embodiment, the Fc-containing polypeptide has one or more of the following properties when compared to a parent Fc-containing polypeptide: increased effector function; increased ability to recruit immune cells; and increased inflammatory properties.

In one embodiment, the Fc-containing polypeptide of the invention comprises or consist of the amino acid sequence of SEQ ID NO:6 or SEQ ID NO:7. In another embodiment, the Fc-containing polypeptide of the invention comprises or consist of the amino acid sequence of SEQ ID NO:6 or SEQ ID NO:7, plus one or more mutations which result in an increased amount of sialic acid when compared to the amount of sialic acid in a parent Fc-containing polypeptide. In another embodiment, the Fc-containing polypeptide of the invention comprises or consist of the amino acid sequence of SEQ ID NO:6 or SEQ ID NO:7, plus one, two, three or four mutations which result in an increased amount of sialic acid when compared to the amount of sialic acid in a parent Fc-containing polypeptide. In one embodiment, the Fc-containing polypeptide of the invention comprises or consist of the amino acid sequence of SEQ ID NO:8 or SEQ ID NO:9.

As utilized herein, the term “pharmaceutically acceptable” means a non-toxic material that does not interfere with the effectiveness of the biological activity of the active ingredient(s), approved by a regulatory agency of the Federal or a state government or listed in the U.S. Pharmacopoeia or other generally recognized pharmacopoeia for use in animals and, more particularly, in humans. The term “carrier” refers to a diluent, adjuvant, excipient, or vehicle with which the therapeutic is administered and includes, but is not limited to such sterile liquids as water and oils. The characteristics of the carrier will depend on the route of administration.

Pharmaceutical formulations of therapeutic and diagnostic agents may be prepared by mixing with acceptable carriers, excipients, or stabilizers in the form of, e.g., lyophilized powders, slurries, aqueous solutions or suspensions (see, e.g., Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In certain embodiments, the Fc-containing polypeptides of the invention can be administered by an invasive route such as by injection (see above). In some embodiments of the invention, the Fc-containing polypeptides of the invention, or pharmaceutical composition thereof, is administered intravenously, subcutaneously, intramuscularly, intraarterially, intraarticularly (e.g. in arthritis joints), intratumorally, or by inhalation, aerosol delivery. Administration by non-invasive routes (e.g., orally; for example, in a pill, capsule or tablet) is also within the scope of the present invention.

In certain embodiments, the the Fc-containing polypeptides of the invention can be administered by an invasive route such as by injection (see above). In some embodiments of the invention, the Fc-containing polypeptides of the invention, or pharmaceutical composition thereof, is administered intravenously, subcutaneously, intramuscularly, intraarterially, intraarticularly (e.g. in arthritis joints), intratumorally, or by inhalation, aerosol delivery. Administration by non-invasive routes (e.g., orally; for example, in a pill, capsule or tablet) is also within the scope of the present invention.

Compositions can be administered with medical devices known in the art. For example, a pharmaceutical composition of the invention can be administered by injection with a hypodermic needle, including, e.g., a prefilled syringe or autoinjector.

The pharmaceutical compositions of the invention may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

The pharmaceutical compositions of the invention may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternately, one may administer the antibody in a local rather than systemic manner, for example, via injection of the antibody directly into an arthritic joint, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

The administration regimen depends on several factors, including the serum or tissue turnover rate of the therapeutic antibody, the level of symptoms, the immunogenicity of the therapeutic antibody, and the accessibility of the target cells in the biological matrix. Preferably, the administration regimen delivers sufficient therapeutic antibody to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med 342:613-619; Ghosh et al. (2003) New Engl. J. Med. 348:24-32; Lipsky et al. (2000) New Engl. J. Med 343:1594-1602).

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is derived from the same species as the animal targeted for treatment, thereby minimizing any immune response to the reagent. In the case of human subjects, for example, chimeric, humanized and fully human Fc-containing polypeptides are preferred.

Fc-containing polypeptides can be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. A total weekly dose is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more (see, e.g., Yang et al., New Engl. J. Med. 349:427-434 (2003); Herold et al., New Engl. J. Med. 346:1692-1698 (2002); Liu et al., J. Neurol. Neurosurg. Psych. 67:451-456 (1999); Portielji et al., Cancer Immunol. Immunother. 52:133-144 (2003). In other embodiments, an Fc-containing polypeptide Of the present invention is administered subcutaneously or intravenously, on a weekly, biweekly, “every 4 weeks,” monthly, bimonthly, or quarterly basis at 10, 20, 50, 80, 100, 200, 500, 1000 or 2500 mg/subject.

As used herein, the terms “therapeutically effective amount”, “therapeutically effective dose” and “effective amount” refer to an amount of an Fc-containing polypeptide of the invention that, when administered alone or in combination with an additional therapeutic agent to a cell, tissue, or subject, is effective to cause a measurable improvement in one or more symptoms of a disease or condition or the progression of such disease or condition. A therapeutically effective dose further refers to that amount of the Fc-containing polypeptide sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity.

### Example 1

### Construction of Anti-TNFα Fc Muteins

The preparation of an Fc with two mutations (F243A/V264A) in an anti-TNF monoclonal antibody in Pichia pastoris was carried out using the sequences and protocols listed below. The heavy and light chain sequences of the parent (wildtype) anti-TNFα antibody are set for the in SEQ ID NOs:1 and 2. The sequence of the heavy chain of the double mutein anti-TNFα antibody is set forth in SEQ ID NO:3. The light chain sequence of the wt and double mutein anti-TNFα antibodies are identical.

The signal sequence of an alpha-mating factor predomain (SEQ ID NOs: 4 and 5) was fused in frame to the end of the light or heavy chain by PCR fusion. The sequence was codon optimized and synthesized by Genscript (GenScript USA Inc., 860 Centennial Ave. Piscataway, N.J. 08854, USA). Both heavy chain and light chain were cloned into antibody expression vector as similar way of constructing anti-HER2 IgG1 and its Fc muteins.

The heavy and light chains with the fused signal sequence of IgG1 and its muteins were cloned under Pichia pastoris AOX1 promoter and in front of S. cerevisiae Cyc terminator, respectively. The expression cassette of the completed heavy and light chains was put together into the final expression vector. Genomic insertion into Pichia pastoris was achieved by linearization of the vector with Spe1 and targeted integration into the Trp2 site. Plasmid pGLY6964 encodes wildtype anti-TNFαIgG1 antibody. Plasmid pGLY7715 endoes the anti-TNF alpha IgG1 F243A/V264A double mutein.Glycoengineered Pichia GFI6.0 YGLY 22834 was the parental host for producing Anti-TNFα Fc muteins. Its genotype is listed as follow: ura5Δ::ScSUC2 och1Δ::lacZ bmt2Δ::lacZ/KlMNN2-2 mnn4L1Δ::lacZ/MmSLC35A3 pno1Δ mnn4Δ::lacZ ADE1:lacZ/NA10/MmSLC35A3/FB8his1Δ::lacZ/ScGAL10/XB33/DmUGT arg1Δ::HIS1/KD53/TC54bmt4Δ::lacZ bmt1Δ::lacZ bmt3Δ::lacZ TRP2:ARG1/MmCST/HsGNE/HsCSS/HsSPS/MmST6-33ste13Δ::lacZ/TrMDS1 dap2Δ::Nat^{R} TRP5:Hyg^{R}MmCST/HsGNE/HsCSS/HsSPS/MmST6-33 Vps10-1Δ::AOX1p_LmST73d. Anti-TNF α Fc mutein expressing plasmid was transformed into YGLY22834 and generated YGLY23423. YGLY23423 was used as the production strain to make alpha 2,6 sialylated anti-TNF α Fc mutein.

The abbreviations used to describe the genotypes are commonly known and understood by those skilled in the art, and include the following abbreviations:
ScSUC2 S. cerevisiae Invertase
OCH1 Alpha-1,6-mannosyltransferase
KlMNN2-2 K. lactis UDP-GlcNAc transporter
BMT1 Beta-mannose-transfer (beta-mannose elimination)
BMT2 Beta-mannose-transfer (beta-mannose elimination)
BMT3 Beta-mannose-transfer (beta-mannose elimination)
BMT4 Beta-mannose-transfer (beta-mannose elimination)
MNN4L1 MNN4-like 1 (charge elimination)
MmSLC35A3 Mouse homologue of UDP-GlcNAc transporter
PNO1 Phosphomannosylation of N-glycans (charge elimination)
MNN4 Mannosyltransferase (charge elimination)
ScGAL10 UDP-glucose 4-epimerase
X833 Truncated HsGalT1 fused to ScKRE2 leader
DmUGT UDP-Galactose transporter
KD53 Truncated DmMNSII fused to ScMNN2 leader
TC54 Truncated RnGNTII fused to ScMNN2 leader
NA₁₀ Truncated HsGNTI fused to PpSEC12 leader
FB8 Truncated MmMNS1A fused to ScSEC12 leader
TrMDS1 Secreted T. reseei MNS1
ADE1 N-succinyl-5-aminoimidazole-4-carboxamide ribotide (SAICAR) synthetase
MmCST Mouse CMP-sialic acid transporter
HsGNE Human UDP-GlcNAc 2-epimerase/N-acetylmannosamine kinase
HsCSS Human CMP-sialic acid synthase
HsSPS Human N-acetylneuraminate-9-phosphate synthase
MmST6-33 Truncated Mouse α-2,6-sailyl transferase fused to ScKRE2 leader
LmSTT3d Catalytic subunit of oligosaccharyltransferase from Leishmania major

### Yeast Transformation and Screening

The glycoengineered GS6.0 strain was grown in YPD rich media (yeast extract 1%, peptone 2% and 2% dextrose), harvested in the logarithmic phase by centrifugation, and washed three times with ice-cold 1 M sorbitol. One to five μg of a Spe1 digested plasmid was mixed with competent yeast cells and electroporated using a Bio-Rad Gene Pulser Xcell™ (Bio-Rad, 2000 Alfred Nobel Drive, Hercules, Calif. 94547) preset Pichia pastoris electroporation program. After one hour in recovery rich media at 24° C., the cells were plated on a minimal dextrose media (1.34% YNB, 0.0004% biotin, 2% dextrose, 1.5% agar) plate containing 300 μg/ml Zeocin and incubated at 24° C. until the transformants appeared.

### Antibody Purification

Purification of secreted antibody can be performed by one of ordinary skill in the art using available published methods, for example Li et al., Nat. Biotech. 24(2):210-215 (2006), in which antibodies are captured from the fermentation supernatant by Protein A affinity chromatography and further purified using hydrophobic interaction chromatography with a phenyl sepharose fast flow resin.

### Generation of α-2,3 Sialyated Anti-TNF Double Mutein Antibody

The reagent identified as “α2,3 SA IgG” corresponds to an anti-TNF antibody having the amino acid sequence of SEQ ID NO:2 and SEQ ID NO:3 produced in the GFI 6.0 strain described above, which was in vitro treated with neuraminidase to eliminate the α2,6 linked sialic acid, and further in vitro treated with α-2,3 sialyltransferase. Briefly, the purified antibody (4-5 mg/me was in the formulation buffer comprising 6.16 mg sodium chloride, 0.96 mg monobasic sodium phosphate dehydrate, 1.53 mg dibasic sodium phosphate dihydrate, 0.30 mg sodium citrate, 1.30 mg citric acid monohydrate, 12 mg mannitol, 1.0 mg polysorbate 80 per 1 ml adjusted to pH to 5.2. Neuraminidase (10 mU/ml) was added to antibody mixture and incubated at 37° C. for at least 5 hrs or until desialylation reached completion. The desialylated material was applied onto CaptoMMC (GE Healthcare) column purification to remove neuraminidase and reformulated in Sialyltransferase buffer (50 mM Hepes pH 7.2 150 mM NaCl, 2.5 mM CaCl2, 2.5 mM MgCl2, 2.5 mM MnCl2) at 4 mg/ml. Mouse α-2,3 sialyltransferase recombinant enzyme expressed in Pichia and purified via his-tag was used for α-2,3 sialic acid extension. The enzyme mixture was formulated in PBS in the presence of Protease Inhibitor Cocktail (Roche™, cat #11873580001) at 1.2 mg/ml. Prior to the sialylation reaction, pepstatin (50 ug/ml), chymostatin (2 mg/ml) and 10 mM CMP-Sialic acid were added to the enzyme mixture followed by sterilization through 0.2 μm filter. One ml of enzyme mixture was added to 10 ml desialylated material. The reaction was carried out at 37° C. for 8 hrs. The sialylation yield was confirmed by mass determination by ESI-Q-TOF. The final material was purified using MabSelect (GE Healthcare) and formulated in the buffer described above and sterile-filtered (0.2 μm membrane). The glycosylation of the final material was analyzed by HPLC based 2-AB labeling method. Approximately 89% of the N-glycans on the polypeptide comprised an oligosaccharide structure selected from the group consisting of NANA₍₁₋₂₎Gal₍₁₋₂₎GlcNAc(2)Man₃GlcNAc₂.

### Example 2

### Anti-Tumor Activity of α-2,3 Sialylated Fc-Containing Polypeptides

In order to determine if α-2,3 linked sialylation of an Fc-containing polypeptide can enhance the effector function of immune cells, the effect of α-2,3 linked SA IgG was determined using the 4T1 tumor cell line.

A mouse mammary tumor cell line 4T1 [ATCC CRL-2539] stably transfected with firefly luciferase [Luc2] was cultured in RPMI-1640 medium supplemented with 10% FBS. Eight-week old female BALB/c mice were implanted on the ventral side with 3×105 4T1-Luc2 cells by subcutaneous route. A week after implantation, the tumors were evaluated by 3-dimensional measurements using Biopticon Tumorlmager and randomized into treatment groups. Groups of five mice each were treated with indicated doses of antibodies in a weekly treatment regimen for 3 consecutive weeks. Tumor volumes were monitored weekly and results analyzed using GraphPad Prism software.

In this model an anti-mouse PD1 antagonistic antibody (generated in-house) was used as a positive control. An isotype antibody and an anti-CD90 (generated in-house) antibody were used as a negative controls.

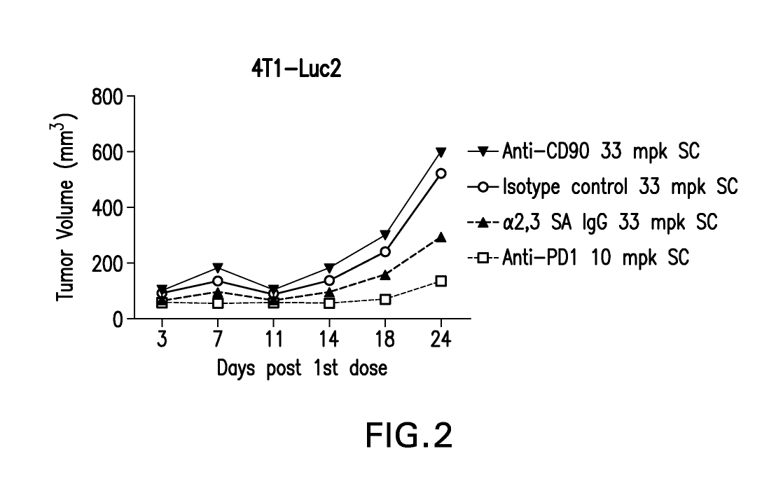

This experiment shows that anti-PD1 treatment activates CD8 cytotoxic responses and suppresses tumor growth whereas anti-CD90 deletes all T lymphocyte subsets and allowes for uncontrolled tumor growth (FIGS. 1-2). α-2,3 SA IgG dramatically reduces tumor volume.

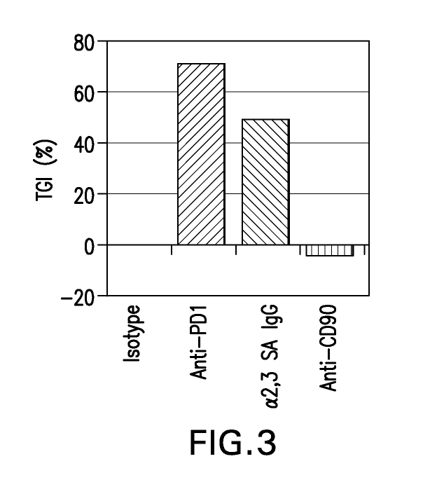

Treatment of subcutaneous 4T1-Luc2 mammary tumor bearing mice with α-2,3 sialylated IgG resulted in a median tumor growth inhibition (“TGI”) of 49% when compared to isotype-treated mice (FIG. 3). Anti-PD1 exhibited strong TGI (69%) while anti-CD90 showed poor TGI. The in-vivo tumor doubling time [TDT] for isotype-treated group was 3.9 days compared to 4.7 days for the α-2,3 sialylated IgG treated group. By this measure, it would take an average of about 34 days for the tumor to reach ˜1600 cubic mm in a α-2,3 sialylated IgG treated animal compared to only about 29 days for an isotype-treated animal.

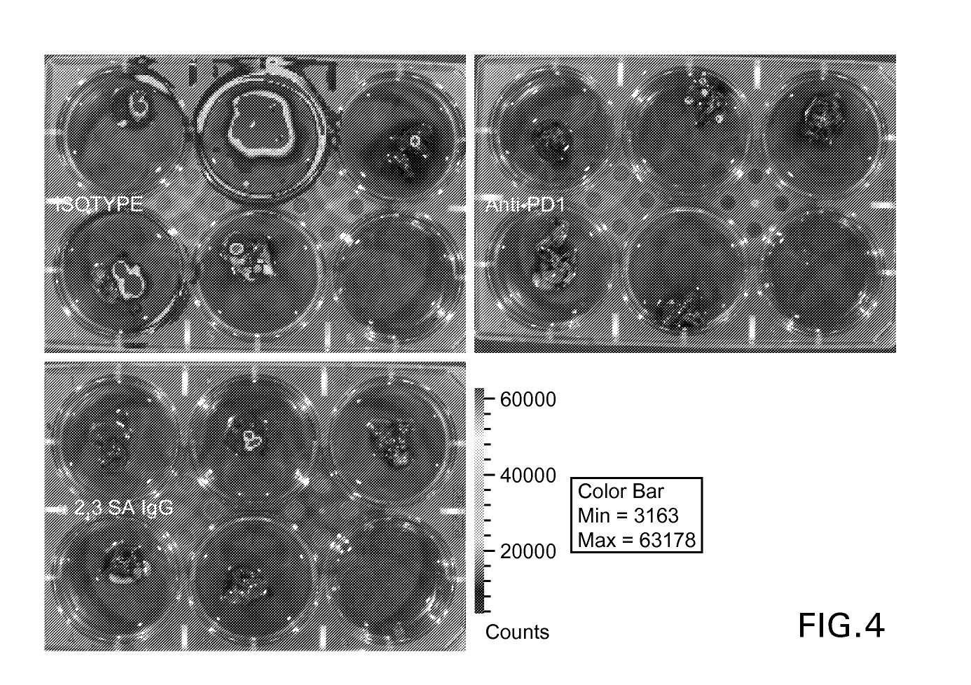

At the end of the study, mice were treated with 150 mg D-luciferin/Kg body weight, and the mice were euthanized after 10 minutes. The lungs were harvested and imaged using IVIS Spectrum [Caliper Life Sciences]. Relative bioluminescence for lung colonization was evaluated using Living Image software. While the tumors in isotype-treated animals exhibit strong tendency to metastasize to the lung [100% metastasis rate], only 20% of the mice show lung colonization when treated with α-2,3 sialylated IgG suggesting an anti-metastatic effect (FIG. 4). Anti-PD1 treatment also exhibits a strong anti-metastatic effect whereas anti-CD90 treatment mice showed remarkable tumor metastasis in all mice (not shown).

### Example 3

### Adjuvant and Anti-Tumor Activity of Anti-CD40 Agonistic Antibody Having an Increased Amount of α2,3 Sialic Acid

CD40 is a member of the tumor necrosis factor receptor (TNFR) super family which is expressed on antigen-presenting cells. CD40 agonists have been shown to trigger immune responses against various tumors and to inhibit the growth of different neoplastic cells, both in vitro and in vivo. It has been shown that an agonistic mAb to CD40, with enhanced binding to Fc gamma receptor JIB on antigen-presenting cells, increases activation of the antigen-presenting cells and thereby promotes an adaptive immune response (Li and Ravetch, Science 333(6045):1030 (2011)). It was proposed that agonistic CD40 antibodies require the coengagement of the inhibitory FcgRIIB, leading to the maturation of DCs promoting the expansion and activation of cytotoxic CD8+ T cells.

In order to study whether an agonistic anti-CD40 mAb with increased Fc gamma receptor IIB binding could benefit from increased α-2,3 sialic acid content at its Fc region, the antibody is modified by introducing mutations F243A/V264A on its Fc region and by expressing the antibody in the GFI6.0 strain. This antibody is then studied in the 4T1 metastatic breast cancer model and/or the murine B-cell lymphoma A20 model for turmor regression and overall long-term animal survival.

The 4T1model is described in Example 2. Briefly a mouse mammary tumor cell line 4T1 [ATCC CRL-2539] stably transfected with firefly luciferase [Luc2] is cultured in RPMI-1640 medium supplemented with 10% FBS. Eight-week old female BALB/c mice are implanted on the ventral side with 3×105 4T1-Luc2 cells by subcutaneous route. A week after implantation, the tumors are evaluated by 3-dimensional measurements using Biopticon TumorImager and randomized into treatment groups. Groups of five mice each are treated with indicated doses of the modified anti-CD40 antibody in a weekly treatment regimen for 3 consecutive weeks. Tumor volumes were monitored weekly and results analyzed using GraphPad Prism software.

In another model, animals are challenged with murine B-cell lymphoma turmor cell A20 and then treated with the modified anti-CD40 antibody. A20 cells are maintained in RPMI with 10% FBS, 1% Pen Strep, 1 mM Sodium Pyruvate, 10 mM HEPES, and 50 μM 2-Mercaptoethanol. BALB/c mice are injected intravenously with either 200 μg of mouse control IgG, or the modified anti-CD40 antibody. One hour later, 2×107 A20 cells are inoculated subcutaneously. Tumor growth and long-term survival for A20 challenged mice are monitered.

### Example 4

### Effect of α2,3 Sialylated Fc Fragment in a Collagen-Antibody Induced Arthritis (AIA) Model

MODEL INDUCTION: AIA (Antibody induced arthritis) is induced with a commercial Arthrogen-CIAe arthritogenic monoclonal antibody (purchased from Chondrex) consisting of a cocktail of 5 monoclonal antibodies, clone A2-10 (IgG2a), F10-21 (IgG2a), D8-6 (IgG2a), D1-2G(IgG2b), and D2-112 (IgG2b), that recognize the conserved epitopes on various species of type II collagen.

ANIMALS: 10 week old B10.RIII male mice which are susceptible to arthritis induction without additional of co-stimulatory factors were used. These animals were purchased from Jackson Laboratory.

CLINICAL SCORING: Paw swelling was measured daily post-induction of arthritis. Each paw was assessed individually and the paw score was added to yield the overall disease score: No swelling=0; Digit swelling=1, Digit and paw selling=2; Digit and paw, with Achilles joint involvement=3; minimum per mouse score=0, maximum score=12.

STUDY DESIGN: Arthritis was induced by passive transfer of 3 mg of anti-CII mAb pathogen cocktail IV on day 0.

Groups of Mice were Treated Subcutaneously with Following Reagents:

| Group/Reagent | Dose |
|---|---|
| α2,3 SA-Fc | 50 mpk |
| Deglycosylated Fc | 50 mpk |
| AIA Control | 50 mpk |
| Naive | 50 mpk |

Group n = 5 for all groups

The reagent identified as “α2,3 Sialyated Fc” corresponds to an Fc fragment comprising the amino acid sequence of SEQ ID NO:9 (but including an additional alanine residue at the 5′ position) produced in a Pichia pastoris strain YGLY31425 having the following geneology: [ura5Δ::ScSUC2 och1Δ::lacZ bmt2Δ::lacZ/KlMNN2-2, mnn4L1Δ::lacZ/MmSLC35A3 pno1Δ mnn4Δ::lacZ, ADE1::lacZ/NA10/MmSLC35A3/FB8, his1Δ::lacZ/ScGAL10/XB33/DmUGT, arg1Δ::HIS1/KD53/TC54, bmt4Δ::lacZ bmt1Δ::lacZ bmt3Δ::lacZ, TRP2::ARG1/MmCST/HsGNE/HsCSS/HsSPS/rSiaT6-33, TRP5::lacZ/MmCST/HsGNE/HsCSS/HsSPS/rSiaT6-33, ADE8::lacZ-URA5-lacZ/TrMDS1/LmSTT3d, TRP2::Sh ble/hFc double mutein (SEQ2), att1Δ::ScARR3]. The reagent was purified using standard in which antibodies are captured from the fermentation supernatant by Protein A affinity chromatography and further purified using hydrophobic interaction chromatography with a phenyl sepharose fast flow resin. The glycosylation of the final material was analyzed by NP-HPLC. Approximately 84% of the N-glycans on the polypeptide comprised bi-sialylated glycans (NANA₂Gal₂GlcNAc₂Man₃GlcNAc₂) with sialic acid linked alpha-2,3 to the penultimate galactose residues.

The reagent identified as “Deglycosylated Fc” corresponds to an Fc fragment comprising the amino acid sequence of SEQ ID NO:9 (but including an additional alanine residue at the 5′ position) produced in Pichia pastoris strain YGLY27893, having the following geneology: [ura5Δ::ScSUC2 och1Δ::lacZ bmt2Δ::lacZ/KlMNN2-2 mnn4L1Δ::lacZ/MmSLC35A3 pno1Δ mnn4Δ::lacZ ADE1:lacZ/NA10/MmSLC35A3/FB8his1Δ::lacZ/ScGAL10/XB33/DmUGT arg1Δ::HIS1/KD53/TC54bmt4Δ::lacZ bmt1Δ::lacZ bmt3Δ::lacZ TRP2:ARG1/MmCST/HsGNE/HsCSS/HsSPS/MmST6-33ste13Δ::lacZ/TrMDS1 dap2Δ::Nat^{R} TRP5:Hyg^{R}MmCST/HsGNE/HsCSS/HsSPS/MmST6-33 Vps10-1Δ:: AOX1p_LmSTT3d TRP2::Sh ble/hFc double mutein (SEQ2)]. The reagent was purified using standard methods in which antibodies are captured from the fermentation supernatant by Protein A affinity chromatography and further purified using hydrophobic interaction chromatography with a phenyl sepharose fast flow resin. The protein obtained was treated in vitro by PNGase to to remove the N-linked glycan.

The group identified as “AIA control” refers to mice that did not receive any treatment (other than the administration of the anti-CH mAb pathogen cocktail to induce AIA).

The group identified as “naive” corresponds to mice that did not receive the anti-CII mAb pathogen cocktail to induce AIA.

All groups of mice were dosed on day 0. The Clinical Score was monitored for 10 days.

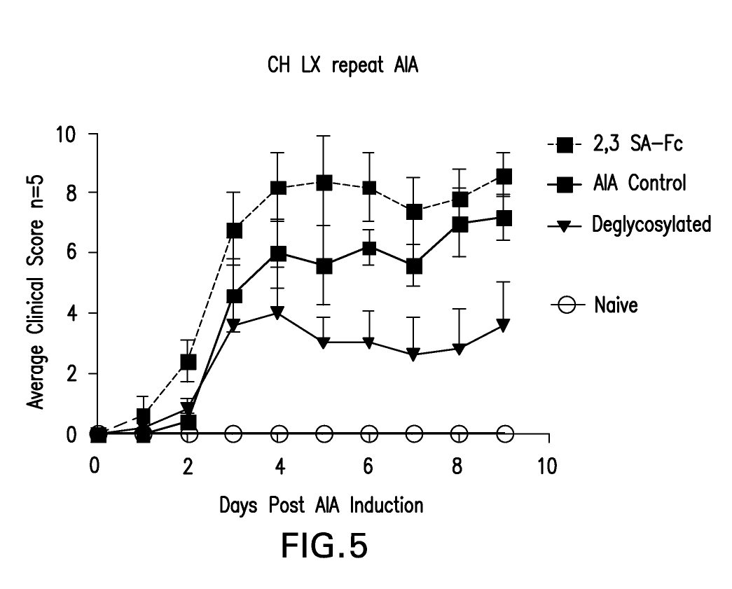

The results of these experiments are shown in FIG. 5. α2,3 sialylated-Fc dramatically enhanced paw swelling and edema in this inflammation model.

SEQUENCE LISTING

| SEQ | | |
|---|---|---|
| ID | | |
| NO: | Description | Sequence |
| 1 | heavy chain | E V Q L V E S G G G L V Q P G R S L R L |
| | amino acid | S C A A S G F T F D D Y A M H W V R Q A |
| | sequence of | P G K G L E W V S A I T W N S G H I D Y |
| | wildtype | A D S V E G R F T I S R D N A K N S L Y |
| | anti-TNF | L Q M N S L R A E D T A V Y Y C A K V S |
| | alpha | Y L S T A S S L D Y W G Q G T L V T V S |
| | antibody | S A S T K G P S V F P L A P S S K S T S |
| | | G G T A A L G C L V K D Y F P E P V T V |
| | | S W N S G A L T S G V H T F P A V L Q S |
| | | S G L Y S L S S V V T V P S S S L G T Q |
| | | T Y I C N V N H K P S N T K V D K K V E |
| | | P K S C D K T H T C P P C P A P E L L G |
| | | G P S V F L F P P K P K D T L M I S R T |
| | | P E V T C V V V D V S H E D P E V K F N |
| | | W Y V D G V E V H N A K T K P R E E Q Y |
| | | N S T Y R V V S V L T V L H Q D W L N G |
| | | K E Y K C K V S N K A L P A P I E K T I |
| | | S K A K G Q P R E P Q V Y T L P P S R D |
| | | E L T K N Q V S L T C L V K G F Y P S D |
| | | I A V E W E S N G Q P E N N Y K T T P P |
| | | V L D S D G S F F L Y S K L T V D K S R |
| | | W Q Q G N V F S C S V M H E A L H N H Y |
| | | T Q K S L S L S P G |
| 2 | Light chain | D I Q M T Q S P S S L S A S V G D R V T |
| | amino acid | I T C R A S Q G I R N Y L A W Y Q Q K P |
| | sequence of | G K A P K L L I Y A A S T L Q S G V P S |
| | anti-TNF | R F S G S G S G T D F T L T I S S L Q P |
| | alpha | E D V A T Y Y C Q R Y N R A P Y T F G Q |
| | antibody | G T K V E I K R T V A A P S V F I F P P |
| | | S D E Q L K S G T A S V V C L L N N F Y |
| | | P R E A K V Q W K V D N A L Q S G N S Q |
| | | E S V T E Q D S K D S T Y S L S S T L T |
| | | L S K A D Y E K H K V Y A C E V T H Q G |
| | | L S S P V T K S F N R G E C |
| 3 | Heavy chain | E V Q L V E S G G G L V Q P G R S |
| | amino acid | L R L S C A A S G F T F D D Y A M |
| | sequence of | H W V R Q A P G K G L E W V S A I |
| | double | T W N S G H I D Y A D S V E G R F |
| | mutein anti- | T I S R D N A K N S L Y L Q M N S |
| | TNF alpha | L R A E D T A V Y Y C A K V S Y L |
| | antibody | S T A S S L D Y W G Q G T L V T V |
| | | S S A S T K G P S V F P L A P S S |
| | | K S T S G G T A A L G C L V K D Y |
| | | F P E P V T V S W N S G A L T S G |
| | | V H T F P A V L Q S S G L Y S L S |
| | | S V V T V P S S S L G T Q T Y I C |
| | | N V N H K P S N T K V D K K V E P |
| | | K S C D K T H T C P P C P A P E L |
| | | L G G P S V F L A P P K P K D T L |
| | | M I S R T P E V T C V V A D V S H |
| | | E D P E V K F N W Y V D G V E V H |
| | | N A K T K P R E E Q Y N S T Y R V |
| | | V S V L T V L H Q D W L N G K E Y |
| | | K C K V S N K A L P A P I E K T I |
| | | S K A K G Q P R E P Q V Y T L P P |
| | | S R D E L T K N Q V S L T C L V K |
| | | G F Y P S D I A V E W E S N G Q P |
| | | E N N Y K T T P P V L D S D G S F |
| | | F L Y S K L T V D K S R W Q Q G N |
| | | V F S C S V M H E A L H N H Y T Q |
| | | K S L S L S P G |
| 4 | Alpha-mating | GAATTCGAAACGATGAGATTTCCTTCAATTTTTACTGCTGTTTTATTCGCAGCATCCT |
| | factor DNA | CCGCATTAGCT |
| | sequence | |
| 5 | Alpha-mating | MRFPSIFTAVLFAASSALA |
| | factor amino | |
| | acid | |
| | sequence | |
| 6 | Fc region | T C P P C P A P E L L G G P S V F L F P |
| | (wt) | P K P K D T L M I S R T P E V T C V V V |
| | | D V S H E D P E V K F N W Y V D G V E V |
| | | H N A K T K P R E E Q Y N S T Y R V V S |
| | | V L T V L H Q D W L N G K E Y K C K V S |
| | | N K A L P A P I E K T I S K A K G Q P R |
| | | E P Q V Y T L P P S R D E L T K N Q V S |
| | | L T C L V K G F Y P S D I A V E W E S N |
| | | G Q P E N N Y K T T P P V L D S D G S F |
| | | F L Y S K L T V D K S R W Q Q G N V F S |
| | | C S V M H E A L H N H Y T Q K S L S L S |
| | | P G |
| 7 | Fc region | E P K S C D K T H T C P P C P A P E L L |
| | (wt) | G G P S V F L F P P K P K D T L M I S R |
| | | T P E V T C V V V D V S H E D P E V K F |
| | | N W Y V D G V E V H N A K T K P R E E Q |
| | | Y N S T Y R V V S V L T V L H Q D W L N |
| | | G K E Y K C K V S N K A L P A P I E K T |
| | | I S K A K G Q P R E P Q V Y T L P P S R |
| | | D E L T K N Q V S L T C L V K G F Y P S |
| | | D I A V E W E S N G Q P E N N Y K T T P |
| | | P V L D S D G S F F L Y S K L T V D K S |
| | | R W Q Q G N V F S C S V M H E A L H N H |
| | | Y T Q K S L S L S P G |
| 8 | Fc region | T C P P C P A P E L L G G P S V F L A P |
| | (DM) | P K P K D T L M I S R T P E V T C V V A |
| | | D V S H E D P E V K F N W Y V D G V E V |
| | | H N A K T K P R E E Q Y N S T Y R V V S |
| | | V L T V L H Q D W L N G K E Y K C K V S |
| | | N K A L P A P I E K T I S K A K G Q P R |
| | | E P Q V Y T L P P S R D E L T K N Q V S |
| | | L T C L V K G F Y P S D I A V E W E S N |
| | | G Q P E N N Y K T T P P V L D S D G S F |
| | | F L Y S K L T V D K S R W Q Q G N V F S |
| | | C S V M H E A L H N H Y T Q K S L S L S |
| | | P G |
| 9 | Fc region | E P K S C D K T H T C P P C P A P E L L |
| | (DM) | G G P S V F L A P P K P K D T L M I S R |
| | | T P E V T C V V A D V S H E D P E V K F |
| | | N W Y V D G V E V H N A K T K P R E E Q |
| | | Y N S T Y R V V S V L T V L H Q D W L N |
| | | G K E Y K C K V S N K A L P A P I E K T |
| | | I S K A K G Q P R E P Q V Y T L P P S R |
| | | D E L T K N Q V S L T C L V K G F Y P S |
| | | D I A V E W E S N G Q P E N N Y K T T P |
| | | P V L D S D G S F F L Y S K L T V D K S |
| | | R W Q Q G N V F S C S V M H E A L H N H |
| | | Y T Q K S L S L S P G |

While the present invention is described herein with reference to illustrated embodiments, it should be understood that the invention is not limited hereto. Those having ordinary skill in the art and access to the teachings herein will recognize additional modifications and embodiments within the scope thereof.

What is claimed:

1) A method of enhancing an immune response in a subject in need thereof comprising administering to the subject a therapeutically effective amount of an Fc-containing polypeptide comprising sialylated N-glycans, wherein the sialic acid residues in the sialylated N-glycans contain α-2,3 linkages, and wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃ GlcNAc₂.

2) The method of claim 1, wherein the subject has, or is at risk of developing, an infectious disease or a neoplastic disease.

3) The method of claim 1 or 2, wherein at least 30%, 40%, 50%, 60%, 70%, 80% or 90% of the N-glycans on the Fc-containing polypeptide comprise an N-linked oligosaccharide structure selected from the group consisting of NANA₂Gal₂GlcNAc₂Man₃GlcNAc₂.

4) The method of any one claims 1-3, wherein the Fc polypeptide is an antibody or antibody fragment.

5) The method of any one claim 1-4, wherein the Fc polypeptide is an antibody fragment consisting essentially of SEQ ID NO:6 or SEQ ID NO:7

6) The method of any one of claims 1-4, wherein the Fc-containing polypeptide is an antibody or antibody fragment comprising or consisting essentially of the amino acid sequence of SEQ ID NO: 6 or SEQ ID NO: 7, plus one or more mutations which result in an increased amount of sialic acid when compared to the amount of sialic acid in the parent polypeptide.

7) The method of claim 6, wherein the Fc-containing polypeptide is an antibody or antibody fragment comprising mutations at positions 243 and 264 of the Fc region wherein the numbering is according to EU index as in Kabat.

8) The method of any one of claims 1-7, wherein said Fc-containing polypeptide has one or more of the following properties when compared to a parent Fc-containing polypeptide:
a) increased effector function
b) increased ability to recruit immune cells, and
c) increased inflammatory properties.

9) A pharmaceutical formulation comprising an Fc-containing polypeptide, wherein the Fc-containing polypeptide comprises sialylated N-glycans, wherein the sialic acid residues in the sialylated N-glycans contain α-2,3 linkages, and wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃GlcNAc₂.

10) The pharmaceutical formulation of claim 9, wherein at least 30%, 40%, 50%, 60%, 70%, 80% or 90% of the N-glycans on the Fc-containing polypeptide comprise an N-linked oligosaccharide structure selected from the group consisting of NANA₂Gal₂GlcNAc₂Man₃GlcNAc₂.

11) The pharmaceutical formulation of any one of claims 9-10, wherein the Fc-containing polypeptide has one or more of the following properties when compared to a parent Fc-containing polypeptide:
(a) increased effector function;
(b) increased ability to recruit immune cells; and
(c) increased inflammatory properties.

12) The pharmaceutical formulation of any one of claims 9-11, wherein the Fc-containing polypeptide is an antibody fragment consisting essentially of SEQ ID NO:6 or SEQ ID NO:7.

13) The pharmaceutical formulation of any one of claims 9-11, wherein the Fc-containing polypeptide comprises or consists of the amino acid sequence of SEQ ID NO:6 or SEQ ID NO:7, plus one or more mutations which result in an increased amount of sialic acid when compared to the amount of sialic acid in a parent polypeptide.

14) The pharmaceutical formulation of claim 13, wherein the Fc-containing polypeptide is an antibody or antibody fragment comprising mutations at positions 243 and 264 of the Fc region wherein the numbering is according to EU index as in Kabat.


SEQUENCE LISTING (9 sequences)

SEQ ID NO: 1 - protein, 450 aa, Artificial Sequence
  Heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, Artificial Sequence
  light chain
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 450 aa, Artificial Sequence
  heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - DNA, 69 bp, Artificial Sequence
  signal sequence
gaattcgaaacgatgagatttccttcaatttttactgctgttttattcgcagcatcctccgcattagct

SEQ ID NO: 5 - protein, 19 aa, Artificial Sequence
  Signal sequence
MRFPSIFTAVLFAASSALA

SEQ ID NO: 6 - protein, 222 aa, Artificial Sequence
  Fc region
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 231 aa, Artificial Sequence
  Fc region
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 202 aa, Artificial Sequence
  Fc region
PKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 231 aa, Artificial Sequence
  Fc region
EPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG